(19) United States
(12) Patent Application Publication
(10) Pub. No.: US 2013/0203725 A1
(43) Pub. Date: Aug. 8, 2013

(54) METHOD TO TREAT SMALL CELL LUNG CANCER

(71) Applicants: Hazel B. Breitz, Seattle, WA (US); Robert De Jager, Rancho Santa Fe, CA (US); Paul L. Weiden, Seattle, WA (US)

(72) Inventors: Hazel B. Breitz, Seattle, WA (US); Robert De Jager, Rancho Santa Fe, CA (US); Paul L. Weiden, Seattle, WA (US)

(73) Assignee: Poniard Pharmaceuticals, Inc., Seattle, WA (US)

(21) Appl. No.: 13/582,922

(22) PCT Filed: Mar. 4, 2011
(86) PCT No.: PCT/US11/27264

§ 371 Date: Feb. 13, 2013

Related U.S. Application Data
(60) Provisional application No. 61/346,777, filed on May 20, 2010.
(60) Provisional application No. 61/345,451, filed on May 17, 2010.
(60) Provisional application No. 61/345,442, filed on May 17, 2010.
(60) Provisional application No. 61/311,169, filed on Mar. 5, 2010.

(51) Int. Cl. A61K 31/555; A61K 45/06

(52) U.S. Cl. CPC A61K 31/555; A61K 45/06
USPC 514/188

(57) ABSTRACT

A method for treatment of small cell lung cancer (SCLC) that does not respond to first-line treatment or that progresses following cessation of first-line organoplatinum chemotherapy is provided that includes the administration of picoplatin, optionally in conjunction with a regimen of best supportive care. Multiple doses of picoplatin can be administered. The picoplatin can also treat SCLC that has metastacized to the brain.

### CROSS-REFERENCE TO RELATED APPLICATIONS

This application claims priority to U.S. provisional application Ser. Nos. 61/311,169, filed Mar. 5, 2010; 61/345,442, filed May 17, 2010; 61/345,451, filed May 17, 2010 and 61/346,777, filed May 20, 2010, all of which are incorporated by reference herein.

### BACKGROUND

Small cell lung cancer (SCLC) accounts for approximately 14% of all lung cancers. In 2004, there were approximately 26,000 new cases in the United States and 51,000 new cases in Europe (Jemal, 2004). The median survival of patients with untreated SCLC is two to four months (Clark, 1998; Glisson, 2003; Davies, 2004). Combination chemotherapy is currently considered standard first-line therapy for SCLC. The most common regimens include platinum (Pt) drugs such as cisplatin or carboplatin and etoposide. Unfortunately, despite the 40-90% response rate to first-line chemotherapy, long-term survival is unusual because patients develop resistance to chemotherapy and relapse (Sundstrom, 2005; Jackman, 2005). Without further, treatment, the overall expected mean survival after disease relapse is two to four months (Huisman, 1999).

At the time of diagnosis, approximately 30% of patients with SCLC will have tumor confined to the ipsilateral chest, mediastinum, and supraclavicular nodes, designated limited disease. Initially 70-90% of these patients will respond to chemotherapy but the recurrence rate is high (75-90%). The median survival time of patients with limited disease ranges from 14 to 20 months with a two-year survival rate of 40%. Even with the addition of radiation therapy to the chest and head, only 6-15% of patients live beyond five years. Patients with more widespread, extensive disease, have an even worse prognosis. Although response rates to initial chemotherapy remain relatively high, i.e., 40-70%, the median survival of 9-11 months is shorter than for patients with limited disease and long-term survival is rare. Fewer than 5% of patients with extensive disease live beyond two years, even with multi-agent, intensive therapy.

Cisplatin, cis-dichlorodiammine platinum (II), the first organoplatinum anticancer drug, was introduced approximately 40 years ago and possesses a wide range of activity against different tumor types and is still widely used in the treatment of various solid tumors in human patients. However, cisplatin also exhibits a number of undesirable side effects, such as kidney damage (nephrotoxicity) and nausea and vomiting. The search for organoplatinum compounds with fewer side effects than cisplatin led to the discovery of carboplatin (cis-diammine-1,1-cyclobutane dicarboxylate platinum (II)), but this compound also exhibits nephrotoxicity and myelotoxicity and is known to cause cumulative dose-related toxicity that results in slow bone marrow recovery. More recently oxaliplatin (trans-1,2-cyclohexanediammine oxalate platinum (II)) was also developed, but this compound possesses significant neurotoxicity, although its nephrotoxicity was reduced relative to carboplatin. Other platinum-containing drugs that are being studied include satraplatin and lobaplatin. In addition to their undesirable side effects, these organoplatinum compounds are not effective against all tumor types and, significantly, tumors can mutate to develop resistance or tolerance to these compounds, resulting in a tumor that can no longer be controlled with these compounds.

Picoplatin or [SP-4-3]-ammine(dichloro)-(2-methylpyridine)platinum(II) (also known as NX 473, ZD0473, or AMD 473) is a new platinum agent that was developed to be effective against platinum-resistant (such as cisplatin-resistant) cell lines, and is intended for the treatment of solid tumors in humans (Raynaud, 1997; Holford, 1998 (both); Rogers, 2002). Like other platinum analogues, picoplatin causes cell death by the formation of covalent cross-links in DNA that interfere with DNA replication and transcription leading to cell death.

Picoplatin and processes for making picoplatin and for using picoplatin in treatment are disclosed and claimed in U.S. Pat. Nos. 5,665,771 (issued Sep. 9, 1997), and 6,518,428 (issued Feb. 11, 2003), and in PCT/GB0102060, filed May 10, 2001, published as WO2001/087313, which are incorporated herein by reference in their entireties.

In Phase I and II second-line studies with picoplatin, responses were seen in several tumor types, including ovarian, prostate cancer, and SCLC. Substantial nephro-, neuro- or ototoxicity have been observed with picoplatin only rarely in animal studies and in Phase I and Phase II trials (Beale, 2003; Treat; 2002; Giaccone, 2002; Gore, 2002).

There is currently no second-line therapy approved by the United States Food and Drug Administration (FDA) for treatment of patients with refractory SCLC. Oral topetecan is indicated for the treatment of relapsed SCLC in patients who responded but who then relapse ≧45 days following cessation of first-line treatment. Patients who are refractory to first-line treatment have an extremely poor prognosis, and there is no approved drug for such patients. The response rate is <10% for any single-agent regimen in refractory patients (Davies, 2004; Murray, 2003; Sundstrom, 2005; NCCN, 2008) The National Comprehensive Cancer Network (NCCN) 2008 guidelines indicate that monotherapy with ifosfamide, paclitaxel, docetaxel, gemcitabine, or topotecan may be used. However, there have been no randomized trials that demonstrate significant response rate or a survival benefit with these agents and their use in this population is often associated with significant drug-related toxicities. There is a high degree of consensus in the published literature that no currently available therapy offers significant benefit to patients who have refractory disease.

Thus, there clearly remains an unmet need for improved chemotherapy for SCLC.

### SUMMARY OF THE INVENTION

The present invention provides a therapeutic method to treat humans afflicted with SCLC by administering an effective amount of picoplatin thereto, preferably in combination with an additional chemotherapeutic agent and/or palliative care so that their lives are extended and/or progression-free survival (PFS) is increased, e.g., as compared to SCLC patients receiving only palliative care. Picoplatin is an effective first- or second-line therapy for SCLC can extend patient lifespan or PFS, over untreated patients or BSC-only patients in second-line therapy, wherein the patient groups are chemotherapy-naïve or have failed first-line chemotherapy, respectively. In such cases, picoplatin is especially beneficial to patients who cannot or do not receive further adjunct chemotherapy following disease progression as described hereinbelow.

The present invention also provides a method for treating SCLC comprising: administering picoplatin to a human patient afflicted with SCLC, that is non-responsive to first-line therapy (stable or progressive through first-line therapy) or has responded to first-line therapy but then has relapsed/progressed within 90 days, after cessation (i.e., after the last dose) of first-line chemotherapy. Such patients are referred to as described herein below, as afflicted with “refractory” SCLC. The non-responsive patient subset fails to respond in that the SCLC remains stable or progresses through or during initial or “first-line” chemotherapy comprising the administration of other Pt-containing anti-cancer agents such as carboplatin and/or cisplatin, and/or non-Pt-containing agents such as CAV, etoposide or irinotecan (see Table 9 below).

This group also includes the subset of refractory patients who initially respond during initial or “first-line” chemotherapy comprising other such agents and then relapse/progress within 45 days (1.5 mos.) after the end of said treatment, and so are considered to be “early-relapsing” patients. Patients who are non-responsive to first-line therapy or who respond to initial therapy but then relapse or progress within 180 days (3-6 months) can also be treated effectively with picoplatin in accord with the protocols set forth herein. Therefore, the present invention also provides a method of treating small cell lung cancer (SCLC) comprising: (a) selecting a human patient afflicted with SCLC, wherein the patient was non-responsive to first-line therapy, such as one carboplatin or cisplatin, or wherein the SCLC responded to said therapy but relapsed within about 180 days of cessation of said therapy; (b) administering an effective amount of picoplatin and, optionally, a regimen of best supportive care (BSC) to said patient, wherein the lifespan and/or progression free survival (PFS) of the patient administered the picoplatin are extended over that of a patient selected in step (a) who receives only a regimen of BSC.

Patients who initially respond, but then relapse within 90-180 days after the cessation of first-line therapy are considered herein to have SCLC that “90-180 progressive” or to have SCLC that is believed to be more sensitive to first-line therapy.

In an embodiment of the present method, the picoplatin-treated patient does not require or receive third-line chemotherapy, following progression of the SCLC, for a period of time, e.g., for up to about one year from progression of the SCLC, or for at least up to about 60 days from progression of the SCLC.

Patients who respond to initial therapy but relapse following 6 months are generally considered to be sensitive to first-line therapy and are retreated with drugs used in first-line therapy. However, such patients can also be treated with the picoplatin regimens disclosed herein, as can chemotherapy-naïve patients.

The present method can result in control of the SCLC and can extend the life (e.g., the median survival time or MST) in non-responsive or early-relapsing patients over the life of such SCLC patients receiving only a regimen of best supportive care (BSC) after failure of first-line therapy, either without the administration of subsequent, adjunct chemotherapy (third-line therapy) to either patient group, or if both patient groups receive subsequent adjunct chemotherapy (and the results are balanced for receipt of third-line therapy).

Picoplatin second-line therapy is especially preferred for patients who do not receive third-line therapy, either due to patient choice or for whom third-line therapy is contraindicated or otherwise not employed or not an option. For patients in the picoplatin treatment group who are no more than, or preferably less than 50 years of age, and/or who exhibit an ECOG performance status of 0 or 1 prior to initiation of treatment(s), the present picoplatin treatment(s) are particularly beneficial.

The present method can also be effective to treat SCLC that has metastasized to remote sites, such as to the brain.

“Disease control” is defined as absence of progression, as assessed by a radiological response (complete or partial, “PR”) or stable disease as discussed hereinbelow. The patients treated with picoplatin, and preferably also receiving BSC, can exhibit an extended progression-free survival (PFS) over the group that does not receive active chemotherapy.

Although not intending to be bound by any particular theory, it is believed that there is a shared mechanism of resistance of SCLC to platinum-containing anti-cancer agents cisplatin and carboplatin as well as to certain non-platinum-containing anti-cancer agents such as etoposide and topotecan involving the overexpression of the cellular efflux pump proteins PgP and MRP1 and the cotransport of the drugs with glutathione in a glutathione-dependent process mediated by the enzyme GST-pil. Picoplatin cannot bind to or otherwise combine with glutathione and is therefore not sensitive to the efflux pump transport/resistance mechanism.

Therefore, it is believed that tumors that are nonresponsive to first-line cisplatin and/or carboplatin and/or etoposide or that who respond but relapse soon after cessation of such first-line therapy, remain or may be substantially sensitive to picoplatin. Furthermore, since topotecan shares the same glutathione-dependent resistance mechanism, tumors including SCLC also do not respond or cease responding to topotecan. Resistance to picoplatin may involve other as-yet undefined mechanisms, perhaps operating at the DNA level. Also, this provides a rationale to use picoplatin in first-line as well as second-line therapy in which these drugs are conventionally employed.

For the tumors that have relapsed after showing a response to cisplatin, or carboplatin, with or without etoposide, the resistance can be reversible after a “Platinum-Free Interval” (“PFI”). This has resulted in the recommendation to retreat patients with the same platin after a Platinum-Free Interval greater than 6 months, e.g. of 6-15 months. These tumors may also be sensitive to topotecan if they have responded but relapsed at least 45 days after first-line treatment.

Due to the long-felt and unmet need to provide any effective therapy following failure of first-line therapy, it was unexpectedly found that administration of picoplatin, optionally with a regimen of BSC, to refractory, non-responsive or to early-relapsing SCLC patients after failure of first-line therapy, e.g., with other Pt-containing anti-cancer drugs, provides a benefit to the patient in terms of an extended lifespan, and progression-free survival over that of patients receiving only BSC following failure of first-line therapy. Therefore, an embodiment of the invention provides a method to extend the period of therapy during which platinum-containing drugs may be effectively employed to treat Pt-susceptable tumors by employing picoplatin as second-line therapy as described herein.

It was also unexpectedly found that administration of picoplatin to non-responsive patients or to both non-responsive and early-relapsing patients provided a survival benefit that was greater that attained by administration of picoplatin to patients who were more sensitive, but who relapsed at 91-180 days from cessation of first-line therapy. In other words, this subset of refractory SCLC patients can be helped by treatment with picoplatin, as are patients who are more responsive to first-line therapy, e.g., are initially more sensitive to other Pt-containing anti-cancer agents.

An embodiment of the present invention thus provides a method for treating SCLC comprising (a) selecting a population of human patients who are afflicted with SCLC that failed to respond to initial treatment (remained stable or progressed through, e.g., progressed after two or more cycles of initial treatment) or that responded to initial treatment and wherein the SCLC then progressed within 45 days or 180 days from the last day of the initial treatment, optionally including patients less than 50 years of age and/or who have an ECOG performance score or 0 or 1 vs. 2; (b) administering an effective amount of picoplatin to said patients; and (c) optionally, concomitantly with step (b) providing to the patients a regimen of best supportive care (BSC), so that the life (OS) or PFS of the picoplatin-treated patients is extended. For example, the life or the PFS of the picoplatin-treated patients can be extended over that of SCLC patients having non-responsive or early-relapsing SCLC who do not receive picoplatin or another anti-cancer agent but receive supportive care alone.

Also, said picoplatin-treated patients may not have received additional or adjunct chemotherapy, for a period of time, following disease progression, e.g., for up to about one year or up to about 60 days from progression, as disclosed herein below. Preferably, in trials, OS in the intent-to-treat population is censored at the time such post-study adjunct chemotherapy is initiated. The BSC can also be initiated or be continued for a period of time following picoplatin therapy. Preferably, the present method can also result in disease control of the SCLC.

In an embodiment of the method according to the invention, the picoplatin can be the only chemotherapeutic anti-cancer agent administered to the patient selected for treatment. In another embodiment, picoplatin is administered to said patient in conjunction with an effective amount of at least one non-platinum anticancer agent.

In a further embodiment of the invention, picoplatin is selected as the initial treatment for SCLC, either administered singly, or in combination with other non-Pt containing anti-cancer agents or therapies, including radiation therapy. Such chemotherapeutic agents include docetaxel, paclitaxel, etoposide, irinotecan, pemetrexed, cyclophosphamide, doxorubicin including liposomal doxorubicin (DL), gemcitabine, vincristine and amrubicin.

Due to the unexpectedly long half-life of picoplatin in human plasma and plasma ultrafiltrate after intravenous or oral administration of picoplatin to human subjects, the picoplatin can be administered prior to the adjunct or second anti-cancer agent so as to provide a period during which the patient is exposed to a therapeutically effective anti-cancer amount of picoplatin and a period during which the patient is exposed to a therapeutically-effective anti-cancer amount of picoplatin and the second agent.

For example, after a rapid distribution phase of about one hour, an intravaneous dose of 120 mg/m² picoplatin was found to exhibit a plasma terminal half-life (t_{1/2}) of about 100-135 hrs. and a plasma ultrafiltrate (PUF) t_{1/2} of about 60-80 hours. The terminal t_{1/2} for orally administered solid picoplatin is about 100-200 hr. in plasma. See e.g., International Application Nos. PCT/US10/00735, filed Mar. 11, 2010, PCT/US08/001,752 and PCT/US08/001,746, filed Feb. 8, 2008 which are incorporated by reference herein.

Therefore, picoplatin can be administered intravenously, followed by a gap of up to about 2 days, preferably up to about 1 hr., e.g., about 50 min.±30 min., during which no anti-cancer drug is administered, followed by administration of, e.g., Doxil®, at about 20-60 mg/m². Following administration of the Doxil® (t_{1/2}=ca. 50 hrs.), the patient can have effective anti-cancer amounts of both picoplatin and Doxil® in their blood until the levels fall below therapeutically-effective anti-cancer levels. Thus, a therapeutically-effective amount of picoplatin can still be present in vivo, after, e.g., the Doxil® concentration has fallen below a therapeutically-effective level. It is believed that this approach will lead to synergistic effects both in anti-cancer efficacy and control or reduction of side-effects (e.g., AEs), due to either agents.

Patients whose SCLC fails to respond to initial treatment as well as those whose SCLC responds to initial treatment and progresses within 45 days of cessation, can advantageously be treated with picoplatin so as to increase their overall survival (lifespan), irrespective of any objective tumor response during picoplatin treatment. In such embodiments, the patients include the subset of those afflicted with SCLC that is highly refractory to the initial previous treatment of the patient (“first-line therapy”) with another platinum-containing drug, such as cisplatin or carboplatin, and/or one or more non-platinum-containing drugs, in that the SCLC does not respond to first-line treatment in that the patient's SCLC remains stable during first-line treatment of at least three cycles (treatment intervals), and until picoplatin treatment, or that progresses during first-line treatment, including SCLC that progresses throughout first-line treatment of at least two cycles (treatment sessions), and continues to progress until initiation of picoplatin treatment.

Picoplatin can be administered in two doses spaced at about 3, 4, 5 or 6 week intervals, preferably at 3 week (21 day) intervals. In one embodiment of the invention, about 60 mg/m²-150 mg/m², or in a second embodiment, preferably about 150 mg/m² of picoplatin is administered in each dose. The dose may be administered orally or parenterally, or via combination of oral and parenteral routes. In one embodiment, the picoplatin doses are administered by intravenous infusion of an aqueous solution of picoplatin. The infusion of one dose is typically carried out over about one to two hours. Preferably, the solution is a physiological salt solution that has been previously adjusted to be isotonic with suitable salts. In one embodiment of the invention, about 0.5 mg/ml of picoplatin is present in the aqueous infusion solution, and contains at least one pharmaceutically acceptable tonicity adjuster, such as NaCl, MgCl₂, CaCl₂, KCl and the like. To achieve the preferred dosing, preferably about 200-300 mg of picoplatin is administered per dose, e.g., per intravenous infusion.

Over the course of treatment of the cancer, 2-15 doses of picoplatin can be administered, with 2-4 doses being typically administered, at intervals of about 21 days (three weeks). Intervals of up to six weeks, e.g., 3-4 weeks, can be employed if, for example, it is necessary to modify the treatment schedule to reduce side-effects.

As used herein, the term “afflicted with SCLC,” is intended to encompass a patient who is afflicted with combined histology SCLC/non-small cell lung cancer, whose cancer has metastasized to sites other than the lung.

As used herein, the term “afflicted with SCLC” also includes patient whose cancer has metastasized to sites other than the lung.

In one embodiment of the present invention a patient afflicted with SCLC, determined to have an absolute neutrophil count of at least 1.5×10⁹/L and a platelet count of at least 100×10⁹/L, a first dose of about 150 mg/m² picoplatin is administered. If the picoplatin is administered intravenously, it is preferably administered over 1-2 hours. A second dose of 150 mg/m² picoplatin is administered to said patient about 21 days after the first dose, and further dosing at this level is continued if hematological parameters remain stable.

Best supportive care (BSC) for SCLC comprises a number of palliative treatments that may also have limited therapeutic efficacy against lung cancer, but are not considered to be curative. For example, in one embodiment of the invention, BSC includes one or more, and preferably all, of irradiation to control symptoms of metastatic cancer, administration of analgesics to control pain, management of constipation, and treatment of dyspnea and treatment of anemia, e.g., by transfusions, so as to maintain hemoglobin levels (i.e., ≧9 g/L). Other features of BSC for lung cancer are set forth below. In an embodiment according to the present invention, picoplatin is administered in conjunction with a regimen of best supportive care. In another embodiment, the picoplatin can be the only chemotherapeutic anti-cancer agent administered to the patient. As lung cancer is predominantly a male disease, the patient can be a male patient. As lung cancer is prevalent in Asian countries, e.g., China, Russia, and Central and Eastern Europe, the patent can be selected from ethnic groups representative of these geographic regions. Preferably, the patient presents for picoplatin treatment with ECOG PS of 0 or 1, and/or is less than 50 years of age. The patient may have stable disease or may have progressive disease at the time that picoplatin treatment is begun.

The present method can further comprise administering an effective anti-emetic amount of a 5-HT₃ receptor antagonist and dexamethasone to the patient prior to step (c).

The present invention also provides method comprising administering a dosage form adapted for intravenous administration of picoplatin comprising, a solution comprising: (a) water; (b) a tonicity adjuster, such as NaCl, in an amount effective to render the solution isotonic; (c) about 0.5 mg/ml dissolved picoplatin, wherein administration of said dosage form is effective to treat SCLC.

Thus, the present invention also provides a method to use picoplatin to prepare a medicament effective to treat SCLC by administering said medicament, orally or parenterally, to a patient afflicted with SCLC, either as first-line therapy, or as second-line therapy, as said therapy is discussed herein, e.g., in combination with BSC, so that the life of the patient or the PFS of the patient is extended. The medicament can be used singly or with adjunct chemotherapies, as described above.

### BRIEF DESCRIPTION OF THE FIGURES

FIG. 1 is a graph comparing overall survival of treated vs. untreated SCLC patients who were refractory or relapsed within 45 days.

FIG. 2 is a graph comparing time to progression of treated vs. untreated SCLC patients.

FIG. 3 is a graph comparing progression-free survival of treated vs. untreated SCLC patients.

FIG. 4 is a graph comparing overall survival of treated vs. untreated SCLC patients who did not receive post-study chemotherapy.

FIG. 5 is FLT PET SUV and MRI tumor burden measurements after picoplatin treatment in a SCLC brain metastasis model. (A), FLT PET SUV monitored during the course of treatment. SUV values were normalized to pre-treatment SUV values for each animal. PET imaging was performed 24 h after each drug administration. Arrows indicate drug dosing days in each group. (B), MR tumor burden of individual mice in the vehicle control group monitored over time. Red line represents the mean tumor burden for the group. Black arrows indicate dosing times. MRI measurements were performed 24 h after each treatment and again after 3 to 4 days (twice per week). (C), MRI tumor burden of individual mice in the picoplatin treatment group monitored over time. The dashed green line represents the mean tumor burden for the picoplatin treatment group. The red line from B is transposed for comparison. Red arrows indicate dosing times. Error bars represent standard error. N=12 per group.

### DETAILED DESCRIPTION OF THE INVENTION

One embodiment of the invention herein provides a method of treatment and a dosage form suitable for treatment of non-responsive and/or early-relapsing refractory SCLC. For example, if the first-line chemotherapy regimen includes administration of cisplatin or carboplatin, and the tumor is non-responsive to that treatment, in that it remains stable or progresses through initial treatment, or that responds to initial treatment, then recurs within 45 days, such a tumor can be effectively treated with picoplatin as described herein. Non-responsive patients or patients who relapse within 45 days are considered to be in a platinum-free interval following cessation of first-line therapy, in that further treatment with Pt-containing anti-cancer agents other than picoplatin is less effective, e.g., may be wholly ineffective.

Picoplatin is a cytotoxic platinum compound with the chemical name of cis-amminedichloro(2-methylpyridine)platinum(II), or alternatively, [SP-4-3]-ammine(dichloro)-(2-methylpyridine)platinum(II). The name “picoplatin” has been designated as the United States Adopted Name (USAN), the British Approved Name (BAN) and the International Nonproprietary Name (INN) for this product. The molecular formula of picoplatin is C₆H₁₀N₂Cl₂Pt with a molecular weight of 376.14. The structural formula of picoplatin is:

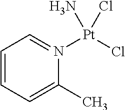

The present invention provides a picoplatin dosage form that comprises a preferably sterile, preferably isotonic, aqueous solution adapted for intravenous (IV) administration. The solution, contains water, picoplatin at a concentration of about 0.3-0.75 mg/mL, e.g., about 0.75-1.0 wt. %, or about 0.5 mg/mL and a tonicity adjuster, such as NaCl. Preferably a preservative is not employed in the solution. The density of the solution is 1.005 g/mL.

TABLE 1
Quantitative Composition Of Picoplatin Intravenous Infusion

| Ingredient | Function |
|---|---|
| Picoplatin | Active Ingredient (0.5 mg/ml) |
| Sodium Chloride USP | Tonicity Adjuster (0.9%) |
| Water for Injection USP | Solvent |

The inventors herein have recognized that administration of picoplatin, for example by intravenous administration, to the population of patients with SCLC that is unresponsive to first-line organoplatinum therapy, or that responded but then progresses within the 45 day period after cessation of the first-line therapy, would be advantageous in terms of inhibiting further progression of the SCLC and/or in prolonging the patients' lives. SCLC that fails to respond to first-line treatment or responds then progresses within 90 days was referred to in Example 2 as “refractory” SCLC. The subset of refractory SCLC that initially responds to first line therapy and then progresses within 45 days is referred to as “early-relapsing” SCLC. Cancer that initially responds to first line therapy, then progresses during the 91-180 day period can also be referred to as non-refractory SCLC.

The picoplatin can be administered in doses ranging from about 60 mg/m² up to about 150 mg/m² per dose, which has been determined to be the maximum tolerated dose for second-line treatment of SCLC, following initial platinum drug therapy. These dosage units refer to the quantity in milligrams per square meter of body surface area.

In another embodiment according to the invention, patients afflicted with SCLC can be treated with picoplatin in conjunction with a regimen of best supportive care (BSC), and BSC can be continued for a period of time after picoplatin therapy has been completed. In an embodiment of the invention, the patients treated with picoplatin and afflicted with progressive disease were not treated with third-line adjunct therapy for up to about one year, e.g., for up to about 60 days from progression. The general guidelines used to provide subjects with BSC are based on the NCCN Guidelines for SCLC and for palliative care (NCCN Palliative Care Guidelines, 2007). In another embodiment, the picoplatin can be the only Pt-containing chemotherapeutic anti-cancer agent administered to the patient selected for treatment, and preferably it is the only anti-cancer agent that is administered to the patient. In a further embodiment, particularly if picoplatin is selected for first-line or initial chemotherapy, it can be used in combination with other non-Pt-containing anti-cancer agents as disclosed, for example, in U.S. patent application Ser. No. 10/276,503, filed Sep. 4, 2003, and incorporated by reference herein. Such agents can include those disclosed as useful in “adjunct chemotherapy,” disclosed herein below.

The invention herein further includes a method of treating SCLC wherein an effective anti-emetic amount of a 5-HT₃ receptor antagonist and dexamethasone are administered to the patient prior to administration of the picoplatin, in order to reduce the side effects of nausea and vomiting that can accompany administration of organoplatinum compounds. An example of a 5-HT₃ receptor antagonist that can be used according to the invention is ondansetron.

### Example 1

### Phase II Study

A Phase II study of picoplatin monotherapy for patients collectively afflicted with SCLC who have non-responsive SCLC, or SCLC that responds to first-line therapy but then progresses within 90 days of completing first-line therapy, or 91-180 day progressive disease, as defined herein, was carried out. A cohort of 77 patients, who had measurable disease, including 44 whose SCLC was unresponsive to first-line organoplatinum chemotherapy (cisplatin, carboplatin or oxaliplatin) and 27 whose SCLC recurred within 90 days after cessation of first-line therapy, plus 6 patients with 91-180 day progressive SCLC, were treated with picoplatin at a dosage of 150 mg/m² given intravenously over a period of 1-2 hours every 21 days. Picoplatin was provided as a sterile isotonic 0.5 mg/mL aqueous solution for IV infusion.

Patients received 1-10 cycles of picoplatin. A median number of dosage cycles of 2, and a mean number of dosage cycles of 3, were administered. Adverse events (AEs) were graded using the NCI CTCAE. The most frequently reported AEs of any severity are shown in Table 2, below. There was no grade 3 or 4 neurotoxicity, ototoxicity, or nephrotoxicity. There were no treatment-related deaths.

TABLE 2
Safety

| | | | Related to Drug |
|---|---|---|---|
| | All Grades (%) | Grades 3/4 (%) | (%) |
| Thrombocytopenia | 49 (67.6) | 37 (48.1) | 49 (63.6) |
| Anemia | 38 (49.4) | 15 (19.5) | 34 (44.2) |
| Neutropenia | 30 (39) | 19 (24.7) | 29 (37.7) |
| Nausea | 21 (27.3) | 1 (1.3) | 17 (22.1) |
| Dyspnea | 12 (15.6) | 3 (3.9) | 2 (2.6) |
| Fatigue | 12 (15.6) | 3 (3.9) | 8 (10.4) |
| Leukopenia | 14 (18.2) | 5 (6.5) | 14 (18.2) |
| Constipation | 11 (14.3) | 1 (1.3) | 6 (7.8) |
| Cough | 9 (11.7) | 1 (1.3) | 1 (1.3) |
| Vomiting | 10 (13.0) | 1 (1.3) | 7 (9.1) |
| Anorexia | 10 (13.0) | 1 (1.3) | 6 (7.8) |
| Asthenia | 8 (10.4) | 2 (2.6) | 2 (2.6) |

Tumor response was assessed every 6 weeks using RECIST criteria. Of 77 patients, three (4%) had partial response (PR), 33 (43%) had stable disease (unconfirmed PR+SD) and 36 (47%) had progressive disease. Disease control rate was 47% in the 77 patients. Median overall survival was 27 weeks (63 of 77 death events; 95% CI=21-33 weeks). The one-year survival rate was 16.9% (95% CI=11-28). Median progression-free survival was 9.1 weeks (71 of 77 progression events; 95% CI=7-12 weeks). Picoplatin monotherapy resulted in median survival that compares favorably with other reported therapeutic options for SCLC and had a reduced toxicity profile.

### Dose Reduction

White blood cell counts (WBC) with differential and platelet counts and hemoglobin are obtained once between Days 11-15 of Cycles 1 and 2 to determine whether or not hematological toxicity has occurred. Subsequent doses for each subject are reduced by 30 mg/m² increments per cycle, up to two reductions, if toxicity is observed. Picoplatin is delayed up to 21 additional days and the dose reduced, if limits for absolute neutrophil count (ANC) and platelet counts are not met or for any other toxicity. Doses of picoplatin can be delayed in the event of unresolved hematological toxicities as described below. Doses of picoplatin are reduced in the event of hematological toxicity in the previous cycle, increased creatinine, or a change in body weight as described below. Once a subject has received a dose reduction, the dose may not be re-escalated. Subsequent treatments will continue at that level unless the toxicity recurs, in which case a further reduction of 30 mg/m² of the reduced dose may be made. Up to two dose reductions will be allowed. If an investigator determines that the degree of dose reduction should be greater than what is contained in these guidelines, investigator discretion shall take precedence to protect the safety of the subject. Similarly, if an investigator determines that a dose reduction should be applied earlier than suggested by these guidelines, investigator discretion shall take precedence to protect the safety of the subject.

The following hematological values must be obtained before picoplatin is administered: absolute neutrophil count (ANC) ≧1.5×10⁹/L; and platelet count ≧100×10⁹/L. If these criteria are not met, then laboratory tests should be measured at a minimum of weekly intervals to see if the required laboratory values are reached. In the event of an absolute neutrophil count less than 0.5×10⁹/L or a platelet count less than 25×10⁹/L, hematology values must be monitored at least twice a week until the neutrophil and platelet counts have improved to above these levels.

A maximum of 21 days is allowed for resolution of the events that do not meet the dosing criteria (i.e., to Day 42 of the cycle). Subjects who do not meet the re-dosing criteria by Day 42 (21 days post planned treatment) should be withdrawn from further treatment for reasons of toxicity.

A dose-reduction of 30 mg/m² is mandatory if any of the following criteria were observed during the previous cycle:

For hematological events: absolute neutrophil count (ANC) <0.5×10⁹/L for at least 5 days; or absolute neutrophil count <1.0×10⁹/L complicated with Grade ≧2 fever; or platelet count <25×10⁹/L; or not reaching a platelet count >100×10⁹/L and absolute neutrophil count >1.5×10⁹/L by Day 21. For non-hematological events (except nausea and vomiting or alopecia): treatment-related Grade 3 toxicity; or any Grade 4 toxicity.

For patients with abnormal serum creatinine, estimated creatinine clearance should be determined. If the calculated creatinine clearance is <60 mL/min, the subject should be monitored to ensure that there is no further deterioration in renal function. If a reduction in creatinine clearance is observed, the dose of picoplatin should be modified according to Table 3. Dose reductions are in the range of 30-60 mg/m² per administration.

TABLE 3

| Calculated creatinine | | |
|---|---|---|
| clearance value | | Dose modification |
| ≧60 | mL/min | recommended dose |
| >40 to <60 | mL/min | reduce by 30 mg/m² |
| >25 to ≦40 | mL/min | reduce by 60 mg/m²* |
| ≦25 | mL/min | discontinue treatment with picoplatin |

*If dose reduction would result in the patient receiving <60 mg/m² of picoplatin, the patient should be taken off study treatment.

A change in weight of 10% or more from that used in the previous calculation of body surface area requires a recalculation in body surface area and appropriate modification of drug dose.

### Example 2

### Phase III study

### Introduction

A Phase III clinical study was carried out to demonstrate median survival superiority of picoplatin monotherapy with best supportive care (BSC) compared to best supportive care alone in non-responsive SCLC patients or in responsive SCLC patients who exhibited progressive disease within 180 days, including refractory and sensitive patients, as defined above.

### Documentation of Disease

Radiological documentation of disease prior to first-line therapy must be available so that the disease status at study baseline can be assessed for protocol eligibility and stratification purposes. The investigator determined eligibility by comparison of chest X-ray or computed tomography (CT) scans obtained prior to, during, and following first-line chemotherapy. During screening, baseline CT or magnetic resonance imaging (MRI) scans were performed for tumor assessment.

The baseline chest and abdomen computed tomography (CT) scans, including bone windows, were used by the radiologist or investigator to confirm radiological status of disease, whether there is measurable disease or not, and to determine the extent of disease at study entry including identification of target lesions, if any, for RECIST response evaluations following subsequent CT scan(s). Measurable disease was not required for study entry, but was required of patients in the radiographically evaluable (RE) population in order to enable the assessment of response rate.

The brain was assessed by a head CT or magnetic resonance imaging (MRI) prior to entry to the study. If brain metastases are identified during the screening exam, treatment with cranial radiation is required prior to randomization. Subjects with symptomatic brain metastases must have been treated with radiotherapy before study entry and must be asymptomatic at the time of baseline evaluations.

### Staging of SCLC

Protocol eligible subjects must have SCLC that is either non-responsive to first-line chemotherapy, or that initially responded to first-line therapy then relapsed within 90 days of the cessation of first-line therapy, or responded to first-line therapy then progressed within 91 to 180 days of completing first-line, platinum-containing chemotherapy.

For the purposes of patient stratification in the trial, subjects were stratified by response to first-line therapy as follows:
Refractory includes subjects whose disease failed to respond to platinum-containing first-line chemotherapy or who responded, then relapsed within 90 days of completing first-line therapy as discussed hereinabove.
Non-refractory includes subjects who initially responded to first-line, platinum-containing chemotherapy and then progressed within 91-180 days after completion of first-line chemotherapy.

Subjects were stratified within geographic region (Europe, India, or South America) and by ECOG performance status [PS; (0 or 1 vs. 2)], as shown in Table 4.

TABLE 4
Eastern Cooperative Oncology Group Performance Status
ECOG Performance Status Scale

| Grade | Description |
|---|---|
| 0 | Normal activity. Fully active, able to carry on all pre- |
| | disease performance without restriction. |
| 1 | Symptoms but ambulatory. Restricted in physically |
| | strenuous activity, but ambulatory and able to carry out |
| | work of a light or sedentary nature (e.g., light housework, |
| | office work). |
| 2 | In bed <50% of the time. Ambulatory and capable of all |
| | self-care, but unable to carry out any work activities. Up |
| | and about more than 50% of waking hours. |
| 3 | In bed >50% of the time. Capable of only limited self- |
| | care, confined to bed or chair more than 50% of waking |
| | hours. |
| 4 | 100% bedridden. Completely disabled. Cannot carry on |
| | any self-care. Totally confined to bed or chair. |
| 5 | Dead. |

There was no time restriction from date of progression until study entry. After stratification, subjects were centrally randomized and assigned 2:1 to receive either picoplatin every 3 weeks plus BSC, or BSC alone.

### Picoplatin Plus BSC Subjects

Prior to each treatment cycle, blood counts, serum chemistry values and calculated creatinine clearance were assessed. The initial dose of picoplatin was 150 mg/m² as a 1-2 hour intravenous infusion on Day 1 of a 21-day cycle.

Subjects who were randomized to the picoplatin plus BSC arm received anti-emetic therapy with a 5-HT₃ receptor antagonist and dexamethasone prior to administration of study drug and received antiemetics following study drug administration, as necessary. All subjects received BSC. White blood cell counts (WBC) with differential and platelet counts were obtained once between Days 11-15 of Cycles 1, 2, and 3 and during any cycle in which a dose reduction was required for hematological toxicity. Picoplatin was delayed up to 21 days, and the dose was reduced, if limits for absolute neutrophil count (ANC) and platelet counts were not met or for any other toxicity.

Follow-up CT scans or other assessments of tumor response were performed every 6 weeks, or after every other cycle while receiving picoplatin, until disease progression. Although 6 cycles of picoplatin are recommended, subjects continued to receive picoplatin beyond 6 cycles as long as they tolerated therapy well and do not have progressive disease.

After discontinuation of picoplatin, all subjects continued to receive BSC, and were to be evaluated every 3 weeks until death, study discontinuation, or the end of the study.

Toxicities were graded using the NCI CTCAE v3. Therapy was delayed up to 21 days (Day 42 of the cycle) if protocol-specified limits for absolute neutrophil count (ANC) and/or platelet count were not met or for any other toxicity.

After discontinuation of picoplatin for any reason, subjects may be treated with other chemotherapy at the investigator's discretion and will then be followed for survival

### BSC Alone Subjects

All BSC alone subjects were evaluated every 3 weeks and continued to receive BSC regardless of disease progression, until death, study discontinuation, or the end of the study. At the time of each visit, a physical examination was done and CBC and serum chemistry values were assessed. Follow-up CT scans were performed every 6 weeks, until disease progression. The delivery of BSC throughout the study, including after disease progression, was monitored.

In the event of disease progression, subjects may be treated with other chemotherapy at the investigator's discretion and will then be followed for survival. Subjects randomized to receive BSC alone did not cross over to receive picoplatin therapy.

### All Subjects

Subjects must have documented radiographic evidence of SCLC. Patients with either measurable or non-measurable disease may be included in this protocol. CT scans at approximately 6 week intervals were used to monitor disease status.

### Measurable Lesions

Measurable disease is the presence of at least 1 measurable lesion. Measurable lesions are those lesions that can be accurately measured in at least 1 dimension with longest dimension ≧20 mm using conventional techniques or ≧10 mm with spiral CT scan. All baseline evaluations were performed as closely as possible to the beginning of treatment and not more than 3 weeks before the beginning of the treatment.

Clinical lesions were only considered measurable when they are superficial (e.g., skin nodules and palpable lymph nodes).

All subjects received treatment with BSC per protocol specified guidelines. This included analgesics, radiation therapy for painful bone metastases, cranial irradiation for brain metastases, radiation for relief of obstructive symptoms from lesions in the chest, nutritional support, and treatment of anemia, dyspnea, infections, paraneoplastic syndromes and anxiety.

The need for BSC interventions was evaluated during visits to the investigator every 3 weeks for all subjects independent of treatment assignment. The general condition of the subjects as well as any AEs was assessed. Lab tests (complete blood count and serum chemistries) were assessed regularly. Prompt palliative interventions to provide BSC for all subjects were prescribed or administered as a result of these evaluations. If a new chemotherapy was introduced, the subject was followed for survival.

Safety was evaluated from the incidence of laboratory and non-laboratory AEs, including SAEs. The severity of all AEs was evaluated according to the National Cancer Institute (NCI) Common Terminology Criteria for Adverse Events (CTCAE) Grading Scale, version 3.

### Inclusion Criteria

To be included in the study, subjects must have met all of the following criteria, listed on Table 5 below.

TABLE 5
Inclusion Criteria

| | |
|---|---|
| 1. | Histological or cytological diagnosis of SCLC (except small |
| | cell adenocarcinoma) or combined SCLC/NSCLC defined as |
| | SCLC mixed with squamous cell carcinoma, |
| | adenocarcinoma, or large cell carcinoma. |
| 2. | One and only 1 prior cisplatin or carboplatin-containing |
| | chemotherapy regimen for SCLC within the scope of the |
| | NCCN Guidelines, as shown below. |
| 3. | Radiologic evidence of SCLC that never responded or |
| | progressed within 90 days after completion of first-line |
| | therapy (refractory); or responded initially to first-line |
| | therapy but progressed between 91 and 180 days after |
| | treatment was completed (progressed within 91 to 180 days). |
| 4. | CT scans of head, chest and abdomen (including adrenals and |
| | full extent of liver) with contrast, preferably within 14 days |
| | prior to randomization (up to 21 days is allowed if |
| | necessary). MRI is acceptable in the case of allergy to |
| | contrast agents. The presence or absence of measurable |
| | disease by RECIST must be documented from the baseline |
| | CT or MRI scan. |
| 5. | Patients with brain metastases must have been treated with |
| | brain irradiation. Only patients with asymptomatic brain |
| | metastases are eligible for this study. |
| 6. | ECOG PS 0, 1 or 2 within 3 days prior to randomization. |
| 7. | Life expectancy of at least 8 weeks within 3 days prior to |
| | randomization. |
| 8. | At least 21 days must have elapsed since the most recent |
| | prior chemotherapy dose, with evidence of hematological |
| | recovery. |
| 9. | At least 14 days must have elapsed since the most recent |
| | prior radiotherapy dose. |
| 10. | At least 14 days must have elapsed since prior surgery except |
| | for the placement of venous access device or bronchoscopy. |
| 11. | Subject must be recovered to ≦ Grade 1 toxicity from all non- |
| | hematological adverse effects of prior therapies (excluding |
| | alopecia). |
| 12. | Age 18 years or over. |
| 13. | ANC ≧1.5 × 10⁹/L. |
| 14. | Platelet count ≧100 × 10⁹/L. |
| 15. | Hemoglobin of ≧90 g/L (9 g/dL) (transfusion permitted to |
| | achieve this hemoglobin). |
| 16. | Aspartate aminotransferase (AST), alanine aminotransferase |
| | (ALT) and lactate dehydrogenase (LDH) levels ≦2.5 times |
| | upper limit of normal (ULN) or ≦5 times ULN if liver |
| | involvement is present. |
| 17. | Bilirubin of ≦1.5 times ULN. |
| 18. | Blood Urea Nitrogen (BUN) ≦1.5 times ULN (hypovolemic |
| | subjects may be hydrated to achieve this BUN). |
| 19. | Creatinine clearance of ≧50 mL/min, as calculated by the |
| | Cockcroft-Gault formula. |
| 20. | Women of childbearing potential must have a negative |
| | pregnancy test (serum or urine). Sexually active couples of |
| | child-bearing potential must agree to use appropriate birth |
| | control methods during chemotherapy and for 3 months after |
| | chemotherapy. |
| 21. | Signed informed consent. |

### Exclusion Criteria

Subjects meeting any of the following criteria were excluded from study participation, as listed on Table 6.

TABLE 6
Exclusion Criteria

| | |
|---|---|
| 1. | Prior radiotherapy that included ≧30% of the bone marrow. |
| 2. | Pleural effusion as the only radiological evidence of SCLC. |
| 3. | Untreated or symptomatic brain or central nervous system |
| | (CNS) metastases. |
| 4. | Grade 2 or higher peripheral neuropathy. |
| 5. | Significant cardiac disease, defined as myocardial infarction |
| | within 3 months prior to randomization, congestive heart |
| | failure classified by the New York Heart Association as Class |
| | III or IV, uncontrolled cardiac arrhythmias, poorly controlled |
| | or unstable angina or electrocardiographic evidence of acute |
| | ischemia. |
| 6. | Serious medical or psychiatric illness that could potentially |
| | interfere with the completion of study treatment according to |
| | this protocol, e.g., active infection, Crohn's disease, |
| | ulcerative colitis, etc. |
| 7. | Use of other investigational drugs within 30 days prior to |
| | randomization. |
| 8. | Breast-feeding. |
| 9. | History of any other malignancy within 5 years, with the |
| | exception of treated non-melanoma skin cancer or carcinoma |
| | in situ of the cervix. |

### Discontinuation of Study Drug

Subjects were to be discontinued from picoplatin for any of the following reasons, listed on Table 7 below.

TABLE 7
Discontinuation Criteria

| | |
|---|---|
| 1. | Documented progressive disease after 1 or more cycles. |
| | Continue with BSC and evaluations. |
| 2. | Symptomatic deterioration prior to CT documented |
| | progression. |
| | Obtain a CT scan to document radiographic disease |
| | status. |
| | Continue with BSC and evaluations. |
| 3. | Unacceptable toxicity |
| | a) Toxicity that does not respond to dosage reductions. |
| | b) Toxicity that delays the next treatment cycle by more |
| | than 21 days (a maximum of 42 days is allowed between |
| | cycles of study drug). |
| | Continue with BSC and evaluations. |
| 4. | Pregnancy or failure to use adequate birth control. |
| | Continue with BSC and evaluations. |
| 5. | Physician decision, i.e., if a change of therapy, prior to CT |
| | documented progression or symptomatic deterioration, would be in |
| | the best interest of the subject. |
| | If change in therapy includes a systemic chemotherapy, |
| | BSC is recommended and subjects should be followed for |
| | survival. |
| | If change in therapy does not include a systemic |
| | chemotherapy, continue with BSC and evaluations. |
| 6. | Patient decision, i.e., if the subject requests discontinuation |
| | or withdraws consent for any reason prior to CT documented |
| | progression. |
| | If patient decides to discontinue treatment with study |
| | drug, continue with BSC and evaluations. |
| | If patient decides to discontinue participation in the study, |
| | BSC is recommended and subjects should be followed for |
| | survival. |
| 7. | Inability to comply with the protocol. |
| | BSC is recommended and subjects should be followed for |
| | survival. |
| 8. | Subject lost to follow-up. |
| 9. | Subject dies. |

Discontinuation from Study

Subjects could be discontinued from participation in the study for any of the reasons listed on Table 8 below. When the subject is discontinued from the study BSC is recommended and subjects should be followed for survival.

TABLE 8
Study Discontinuation

1. Decision to start a new therapy that includes systemic chemotherapy.
2. Patient decision, i.e., if the subject requests discontinuation or
withdraws consent for any reason.
3. Inability to comply with the protocol.
4. Subject lost to follow-up.
5. Subject dies.

“Systemic chemotherapy” or “additional chemotherapy” following picoplatin therapy or following a regimen of BSC as referenced in Tables 7 and 8 can also be referred to as “third-line therapy.” Such therapy can include further treatment with drugs used in first-line therapy, including carboplatin and cisplatin. Carboplatin can be used as a single agent, or combined with a second, non-platinum containing agent such as cyclophosphamide (Cy) optionally with adriamycin and/or vincristine and/or epirubicin, a taxane, taxol, irinotecan, lomustine, gemcitabine, vincristine, (e.g., CEV), etoposide (Ep) or any combination thereof. Cisplatin can be used in combination with irinotecan, Cy, lomustine, gemcytabine (Gem), nimoliezumab, Ep, taxane, taxol, navelbine (Nv) and/or Ep. Other chemotherapeutic agents used in third-line therapy include doxorubicin (singly and with Cy and, optionally vincristine (Vn), Nv, FAU, lomustine, and/or methotrexate), irinotecan, lomustine, gefitinib, hydrazine sulfate, methotrexate, taxanes (e.g., docetaxel and/or paclitaxel), irinotecan, topotecan (and other topoisomerase I or II inhibitors), nimotuzumab (e.g., nimotuzumab in combination with cisplatin), cetuximab, gemcytabine (Gem), vinorelbine, optionally in combination with etoposide or Ep alone. The agents provided herein, or any agent useful to treat cancer, can be combined or used alone for therapy, for example, Vn can be administered in combination with adriamycin; cyclophosphamide can be administered in combination with adriamycin and vincristine (CAV); cyclophosphamide can be administered with doxorubicin and Ep (CDE) and optionally lomustine (CCNU & CDE); cyclophosphamide can be administered with epirubicin and vincristine; navelbine can be administered in combination with Ep; or cyclophosphamide can be administered with vincristine.

Such therapies can increase the life expectancy of certain patients beyond that achievable with second-line picoplatin and/or BSC, used without third-line therapy. However, such chemotherapy may be contraindicated in certain patients, or the patient may choose not to receive additional chemotherapy.

### First-Line Chemotherapy for SCLC

All of the patients had received a first-line, platinum-containing therapy consistent with NCCN Guidelines (Table 9).

TABLE 9
Principles of Chemotherapy for Small Cell Lung Cancer According to
the NCCN Practice Guidelines in Oncology, v.1.2006

Extensive Stage Disease
Cisplatin: 75 mg/m² Day 1 and Etoposide: 100 mg/m² Days 1, 2,
3, x 4-6 cycles
Carboplatin: AUC 6 Day 1 and Etoposide: 100 mg/m² Days 1, 2, 3,
x 4-6 cycles
Cisplatin: 60 mg/m² Day 1 and Irinotecan: 60 mg/m² on Days 1,
8, 15, x 4-6 cycles
Cisplatin: 80 mg/m² Day 1 and Etoposide: 80 mg/m² Days 1,
2, 3, x 4-6 cycles
Cisplatin: 25 mg/m² Days 1, 2, 3 and Etoposide: 100 mg/m² Days 1,
2, 3, x 4-6 cycles
Limited Stage Disease, in Association with Chest Radiation*
Cisplatin: 60 mg/m² Day 1 and Etoposide: 120 mg/m² Days 1,
2, 3 x 4 cycles.*
or
Carboplatin: AUC 6 Day 1 and Etoposide: 100 mg/m² Days 1,
2, 3 x 4 cycles.
*This lower dose of cisplatin is recommended for limited stage disease only when concurrent radiation therapy is administered. Otherwise, the doses of chemotherapy should be those recommended for extensive disease. For purposes of protocol eligibility, carboplatin doses of AUC = 5 would be acceptable (although are not recommended or preferred) and etoposide may have been given orally on Days 1-3 at a dose 2x that of the recommended intravenous route.

All of the patients had received cisplatin- and/or carboplatin-based first-line therapies.

Patients with progressive disease must have had at least 2 cycles of first-line therapy to be considered refractory.

Patients with stable disease must have had a least 3 cycles of first-line therapy to be considered refractory.

### Randomization

Subjects were randomized to receive either picoplatin plus BSC or BSC alone using a central 2:1 blocked and stratified randomization scheme. Each subject will be stratified within to the subject's geographic region; by the subject's response to first-line therapy; and by ECOG PS, as described on Table 10 below.

TABLE 10
Stratification of Subjects

| | |
|---|---|
| 1. | Geographic region |
| | Europe |
| | India |
| | South America. |
| 2. | Response to first-line therapy |
| | Response to first-line therapy will be determined by consideration |
| | of radiological examinations from diagnosis until screening for this |
| | protocol. For patients with measurable disease, RECIST should be |
| | utilized to define response and progression. For patients without |
| | measurable disease, new lesions are evidence of progression. |
| | Otherwise, progression of previously known lesions, or response of |
| | known lesions to first-line therapy to be acceptable must be “very |
| | obvious” on the radiological examinations. If disease progression |
| | is in any way equivocal, subjects should be categorized as having |
| | stable disease. |
| | Refractory = patients whose disease failed to respond to first- |
| | line, platinum-containing chemotherapy or who experienced a |
| | relapse within 90 days after completion of first-line, platinum- |
| | containing chemotherapy. This includes patients who were |
| | stable after more than three cycles of first-line chemotherapy |
| | or those who responded after at least two cycles of first-line |
| | chemotherapy and then progressed within 90 days of the |
| | completion of first-line chemotherapy. |
| | Non-refractory = patients who initially responded to first-line, |
| | platinum-containing chemotherapy, but relapse between 90 |
| | and 180 days of completion of first-line, platinum-containing |
| | chemotherapy. |
| 3. | ECOG PS |
| | 0 or 1 |
| | 2. |

The Demographics and Prior Treatment of the enrolled population is given in Table 11, below:

TABLE 11

| | Picoplatin + | | BSC-Alone | |
|---|---|---|---|---|
| | BSC (n = 268) | | (n = 133) | |
| Age at Screening [years (n)] | | | | |
| Mean (Std) | 57.3 | (9.23) | 57.4 | (7.65) |
| Median (Min, Max) | 57.5 | (29, 80) | 58.0 | (27, 73) |
| Gender [n (%)] | | | | |
| Male | 225 | (84.0%) | 110 | (82.7%) |
| Female | 43 | (16.0%) | 23 | (17.3%) |
| Ethnic Origin [n (%)] | | | | |
| Caucasian | 236 | (88.1%) | 118 | (88.7%) |
| Asian/Non-Caucasian/Hispanic | 32 | (11.9%) | 15 | (11.3%) |
| ECOG Performance Status [n (%)] | | | | |
| 0, 1 | 227 | (84.7%) | 114 | (85.7%) |
| 2 | 41 | (15.3%) | 19 | (14.3%) |
| Platinum Containing Chemotherapy | | | | |
| [n (%)] | | | | |
| Cisplatin | 189 | (70.5%) | 91 | (68.4%) |
| Carboplatin | 60 | (22.4%) | 32 | (24.1%) |
| Both (Cisplatin + Carboplatin) | 19 | (7.1%) | 10 | (7.5%) |
| Prior Radiation [n (%)] | | | | |
| Chest | 55 | (71.4%) | 36 | (75%) |
| Brain | 33 | (42.9%) | 17 | (35.4%) |
| Bone | 8 | (10.4%) | 2 | (4.2%) |

### Picoplatin

### Dose

This was an open-label study with a blinded randomization scheme. All subjects randomized to receive picoplatin plus BSC received 150 mg/m² of picoplatin on Day 1 of the first 21-day cycle administered over 1-2 hours. Subsequent doses for each subject may be reduced by 30 mg/m² decrements per cycle for toxicity as specified below. The dose administered must be within 5% of the target dose. If reduced, the picoplatin dose was not subsequently increased for any individual subject.

The starting dose was based on the body surface area (BSA) calculated from the height and weight of the subject at baseline. Body surface area was calculated using the actual weight of the subject to one (1) decimal place. BSA was recorded to two decimal places for calculating dose of study drug. The equation by Mosteller is being used, as follows:

$$B S A ( {m}^{2} ) = \sqrt{\frac{HEIGHT ( cm ) \times WEIGHT ( kg )}{3600}}$$

Subsequent cycles used the BSA calculated for the starting dose. If a subject's weight changes by 10% or more, the BSA was recalculated and the dose adjusted accordingly.

### Handling and Preparation of Picoplatin

Picoplatin was provided as a sterile isotonic 0.5 mg/mL aqueous solution for IV infusion packed in neutral (Type I) glass injection vials with a nominal volume of 200 mL. The vials are sealed with ETFE copolymer-coated chlorinated butyl rubber stoppers and flip-off crimp seals. The weight per mL for the 0.5 mg/mL formulation is 1.005 g.

Picoplatin vials were supplied to the investigational site in individually packed containers to protect the solution from light. The cardboard containers were temperature monitored during shipment to each clinical site. Each vial is intended for single-use only.

### Stability and Storage

The assigned shelf life of picoplatin 0.5 mg/mL in vials is at least 24 months when stored under the conditions defined by the USP of controlled room temperature, 20 to 25° C. USP controlled room temperature conditions allow for temperature excursions that are generally experienced in pharmacies, hospitals and warehouses, between 15° and 30° C. Provided the temperature usually remains within the allowed range, transient deviations from 4° C. to 40° C. are permitted as long as they do not exceed 24 hours in duration. The drug must be protected from light during storage.

### Administering Picoplatin

Picoplatin was supplied as a ready-to-use formulation. The contents of the vials were transferred to a suitable bag for administration. Compatibility of the formulation with ethylene vinyl acetate (EVA) infusion bags, polyvinyl chloride (PVC) infusion tubing and polypropylene syringes is established when the materials are protected from light. The compatibility of the formulation with typical administration sets has been set as 8 hours in a covered infusion bag.

The product is highly light-sensitive and the bag must be protected from light during preparation and administration. The product should not be exposed to ambient light for more than 1 hour. As with other platinum complexes, contact with aluminum should be avoided.

There is no preservative or bacteriostatic agent present in the picoplatin formulation. Therefore, picoplatin was to be transferred under aseptic conditions. The solution was to be completely used or discarded within 8 hours of removal from the vial.

Although pretreatment hydration is not required, picoplatin is to only be given to subjects who have an adequate balance of fluid and electrolytes.

Picoplatin was administered by peripheral vein or central line. A 1 hour administration for picoplatin was recommended, although this may be extended to 2 hours at the discretion of the physician if clinically indicated (e.g., concern for volume overload). Concurrent administration of intravenous (IV) fluids, (e.g., normal saline), is not required.

### Anti-Emetic Therapy

All subjects receiving picoplatin received anti-emetic therapy with ondansetron 8 mg IV (or comparable 5-HT₃ inhibitor) plus dexamethasone 8 or 12 mg IV or orally (or equivalent corticosteroid) immediately prior to chemotherapy. Subjects should also have received anti-emetic therapy for several days following treatment as needed, which may include oral lorazepam (Ativan®), prochlorperazine (Compazine®) or similar medications, as clinically indicated for breakthrough nausea and/or vomiting. If vomiting is not controlled with these measures, the dose of ondansetron was to be increased for the second cycle. If still not controlled, aprepitant may be added beginning the third cycle.

### Number of Cycles

Although 6 cycles of therapy were recommended, subjects continued treatment cycles for as long as they did not progress and as long as they continued to tolerate therapy. Subjects were removed from treatment in the event of disease progression or unacceptable toxicity.

### Picoplatin Dose Modifications

### Dose Delays and Reduction

Doses of picoplatin were to be delayed in the event of unresolved toxicities as described in this section and in Table 12 below.

TABLE 12
Dose Modifications

| Observations | | Resolution |
|---|---|---|
| Platelet count <100 × 10⁹/L at Day 42 | → | Discontinue picoplatin. |
| ANC count <1.5 × 10⁹/L at Day 42 | | |
| Platelet count <100 × 10⁹/L at Day 21 | → | Delay until recovery |
| ANC count <1.5 × 10⁹/L at Day 21 | | up to Day 42. |
| Hematological events: | → | Reduce picoplatin |
| Platelet count <25 × 10⁹/L at any time | | Reduce picoplatin dose |
| Platelet count <100 × 10⁹/L at Day 21 | | by 30 mg/m² from |
| ANC count <1.5 × 10⁹/L at Day 21 | | dose administered in |
| ANC count <0.5 × 10⁹/L for at least 5 days | | the previous cycle. For |
| ANC count <1.0 × 10⁹/L complicated with | | example, for the first |
| Grade ≧2 fever (>39° C.) | | dose reduction, 120 |
| Hemoglobin <65 g/L (6.5 g/dL) | | mg/m² picoplatin of |
| Hemoglobin <80 g/L (8.0 g/dL) on Day 21 | | study drug will be |
| Renal Dysfunction: | → | Reduce picoplatin |
| Creatinine Clearance <50 mL/min | | |
| Change in Body Weight: | → | Modify picoplatin dose |
| Change in body weight ≧10% | | using recalculated |
| | | BSA |

A maximum of a 21-day delay was allowed for resolution of the events that do not meet the dosing criteria (i.e., to Day 42 of the cycle). Subjects who do not meet the re-dosing criteria by Day 42 (21 days post planned treatment) were to be withdrawn from further treatment with study drug for reasons of toxicity, but should have continued on study receiving BSC.

### Dose Reductions

The dose of picoplatin was to be reduced by 30 mg/m² decrements in the event of hematological toxicity in the previous cycle, decreased renal function, or significant non-hematological toxicity.

Once a subject has had a dose reduction, the dose must not be re-escalated. Subsequent treatments will continue at that reduced dose level unless the toxicity recurs, in which case a further reduction of 30 mg/m² of the reduced dose may be made.

### Required Hematological Values Prior to Picoplatin Treatment

The following hematological values were obtained before picoplatin is administered: (a) ANC ≧1.5×10⁹/L; (b) Platelet count ≧100×10⁹/L; and Hemoglobin ≧80 g/L (8.0 g/dL).

If these criteria are not met, then laboratory tests were to be repeated at a minimum of weekly intervals to see if the required laboratory values were reached. In the event of an absolute neutrophil count less than 0.5×10⁹/L or a platelet count less than 25×10⁹/L, hematology values were to be monitored at least three times a week until the neutrophil and platelet counts have risen above these levels.

A maximum of a 21-day delay was allowed for resolution of toxicity (hematological or non-hematological) that did not meet the dosing criteria (i.e., to Day 42 of the cycle). Subjects who did not meet the re-dosing criteria by Day 42 (21 days post planned treatment) were to be withdrawn from further picoplatin treatment for reasons of toxicity, but should continue receiving BSC on study.

### Required Dose Reduction

A dose-reduction of 30 mg/m² was mandatory if any of the following criteria listed on Table 13 were observed during the previous cycle.

TABLE 13
Dose Reduction

Hematological events:
ANC <0.5 × 10⁹/L for at least 5 days
ANC <1.0 × 10⁹/L complicated with Grade ≧2 fever (>39° C.)
Platelet count <25 × 10⁹/L
Hemoglobin <65 g/L (6.5 g/dL)
Platelet count <100 × 10⁹/L on Day 21
ANC <1.5 × 10⁹/L on Day 21
Hemoglobin <80 g/L (8.0 g/dL) on Day 21
Non-hematological events (except alopecia):
Treatment-related Grade 3 toxicity
Any Grade 4 toxicity
Grade 3 or 4 nausea or vomiting while receiving recommended anti-
emetic treatment
Decreasing renal function:
If a reduction in creatinine clearance is observed, the dose of picoplatin
should be modified according to Table 14, below.

TABLE 14
Dose Reductions for Picoplatin vs.
Calculated Creatinine Clearance

| Calculated Creatinine | | |
|---|---|---|
| Clearance Value | | Dose Modification |
| ≧50 | mL/min | None |
| >35 to <50 | mL/min | Reduce by 30 mg/m² |
| >25 to ≦35 | mL/min | Reduce by 60 mg/m² |
| ≦25 | mL/min | Discontinue treatment with picoplatin |

### Management of Potential Toxicity

Picoplatin had an excellent safety profile with respect to potential neurotoxicity and nephrotoxicity. However, subjects given picoplatin were observed for the possible development of these events.

Supportive-care measures and symptomatic treatment for any drug-related toxicity was initiated, if clinically indicated.

Picoplatin was only to be given to subjects who have an adequate balance of fluid and electrolytes. Hypovolemic subjects were to be rehydrated and serum creatinine and BUN levels repeated. If reduced creatinine clearance was observed, dose reduction was required.

### Hematological Toxicity

Hematological toxicity was to be managed first, with supportive clinical care as appropriate, and secondly, with subsequent dose reductions, as detailed above.

Platelet transfusions were recommended in the event of a platelet count of less than 50×10⁹/L associated with clinical bleeding or a platelet count of less than 10×10⁹/L in the absence of clinical bleeding.

After an episode of severe neutropenia, dose reduction of picoplatin was the primary therapeutic option. Febrile neutropenia is uncommon following picoplatin. Treatment with hematopoietic colony stimulating factors (e.g., G-CSF) is permitted in subjects at high risk for infection-associated complications, as summarized in the NCCN guidelines. If colony stimulating factors are used, the blood count was to be monitored every 3 days and continued only if ANC remains less than 0.5×10⁹/L. Blood transfusions, for all subjects, or erythropoietin, only for subjects randomized to receive picoplatin, can be used for the treatment of anemia.

### Concomitant Medications

Concomitant medications that may contribute to bleeding episodes (e.g., acetyl salicylate) were to be avoided, consistent with standard clinical practice.

### Best Supportive Care (BSC)

The following general guidelines were utilized to provide subjects with BSC and are based on the NCCN Guidelines for SCLC and for palliative care, version 2 (2006), as shown on Table 15 below.

TABLE 15
BSC

| | |
|---|---|
| 1) | Analgesia |

| | |
|---|---|
| a. | Pain assessment with prescriptions for non-narcotic or narcotic |
| | analgesics, as required. |
| b. | Management of toxicities from analgesic medication including |
| | constipation, nausea and/or gastritis. |

| | |
|---|---|
| 2) | Radiation therapy |

| | |
|---|---|
| a. | Palliative radiation therapy for painful bone metastases |
| | or impending fractures. |
| b. | Cranial irradiation for brain metastases. |
| c. | Local radiotherapy to chest lesions for symptomatic or clinically |
| | significant disease. |

| | |
|---|---|
| 3) | Treatment of dyspnea |

| | |
|---|---|
| a. | Thoracentesis or pleurodesis of pleural effusion. |
| b. | Local radiation therapy if relief of obstruction appears feasible. |
| c. | Bronchodilators. |
| d. | Treatment of pericardial effusion. |
| e. | Oxygen therapy. |
| f. | Opioids for cough and dyspnea. |

| | |
|---|---|
| 4) | Treatment of infections |

| | |
|---|---|
| a. | Antibiotics will be administered for pneumonia, |
| | bronchitis and other infections, as required. |

| | |
|---|---|
| 5) | Treatment of symptoms associated with paraneoplastic syndromes |

| | |
|---|---|
| a. | Fluid restriction, saline infusion and/or demeclocycline for |
| | hyponatremia related to inappropriate antidiuretic hormone |
| | secretion. |
| b. | Adrenal suppression for Cushing's syndrome. |
| c. | Management of neurological impairment. |

| | |
|---|---|
| 6) | Spinal cord compression |

| | |
|---|---|
| a. | Physical examination and radiographic evaluation if symptoms |
| | suggest impending spinal cord compression. |
| b. | Appropriate treatment for spinal cord compression. |

| | |
|---|---|
| 7) | Nutritional support |

| | |
|---|---|
| a. | Nutritional support, as required. |

| | |
|---|---|
| 8) | Management of depression, anxiety and stress |

| | |
|---|---|
| a. | Management of depression, anxiety and/or stress, as required. |

| | |
|---|---|
| 9) | Management of anemia |

| | |
|---|---|
| a. | Maintain hemoglobin in an acceptable range |
| | (i.e., 90-110 g/L) with blood transfusion for all subjects, or |
| | erythropoietin may be used only for subjects receiving |
| | picoplatin. |

The frequency of clinical events that might merit intervention with BSC, as outlined in the categories listed above, was monitored in order to ensure that equally intense and diligent supportive care is provided to all subjects regardless of treatment assignment.

### Study Assessments

### Screening

Screening procedures are listed on Table 16 below. All screening procedures were to be completed within 14 days prior to randomization, except as noted.

TABLE 16
Screening Procedures

| | |
|---|---|
| 1. | General medical history. |
| 2. | History of SCLC: |
| | Documentation of stage at diagnosis; including the |
| | diagnostic tests and their results used to determine the |
| | stage at diagnosis. |
| | Prior chemotherapy regimens. |
| | Prior radiation including fields, total dose, fractionation |
| | scheme. |
| | Key results from all radiological examinations or other |
| | studies conducted between diagnosis and baseline CT |
| | scans for this protocol. |
| 3. | Complete physical exam. |
| 4. | Vital signs including pulse, blood pressure |
| | (systolic/diastolic), respiratory rate, and temperature. |
| 5. | Height (cm). |
| 6. | Weight (kg, recorded with 1 decimal place). |
| 7. | Body surface area (to 2 decimal places) calculated using |
| | actual body weight, according to the following formula |
| | $BSA ( {m}^{2} ) = \sqrt{\frac{Height ( cm ) Weight ( kg )}{3600}}$ |
| | The calculation can also be made using the following |
| | website: www.thedrugmonitor.com/BSA-calculator.html |
| 8. | ECOG PS |
| 9. | Record of concomitant medications. |
| 10. | Clinical laboratory tests: |
| | White blood cell count (WBC) with differential, |
| | hemoglobin (Hgb), and platelet count. |
| | Serum chemistry: BUN, creatinine, glucose, sodium, |
| | potassium, chloride, bicarbonate, calcium, phosphorus, |
| | uric acid, total bilirubin, alkaline phosphatase, LDH, |
| | serum glutamic oxaloacetic transaminase (SGOT)/AST, |
| | serum glutamic pyruvic transaminase (SGPT)/ALT, |
| | albumin, total protein, and magnesium |
| | Calculated creatinine clearance Urinalysis: dipstick, with |
| | microscopic examination in the event of an abnormal |
| | reading on the dipstick. |
| | Pregnancy test for females of childbearing potential |
| | (either serum or urine). |
| 11. | Electrocardiogram. May be omitted if a prior study is |
| | available from the preceding 30 days. |
| 12. | Tumor evaluation: |
| | CT of head, chest and abdomen (including entire liver |
| | and adrenal glands) with contrast. If the subject is unable |
| | to tolerate contrast, both chest CT without contrast to |
| | evaluate lung parenchyma and MRI with contrast to |
| | evaluate chest and abdomen are to be performed. |
| | CT/MRI scans need not be repeated if studies are |
| | available from the preceding 21 days. |
| | Documentation of disease as measurable or non- |
| | measurable, as defined by RECIST. Measurable disease |
| | is defined as at least 1 target lesion at screening. All |
| | measurable lesions, up to a maximum of 5 lesions per |
| | organ and 10 lesions in total, representative of all |
| | involved organs, should be identified as target lesions and |
| | the longest dimension (LD) measured and recorded at |
| | baseline. Target lesions should be selected based on their |
| | size (lesions with the longest dimensions) and their |
| | suitability for accurate repeated measurements. Target |
| | lesions must be outside the fields of prior radiotherapy or, |
| | if within prior radiation fields, must be shown to be |
| | enlarging, or a new lesion. The sum of the longest |
| | dimension (SLD) for all target lesions will be calculated |
| | and reported as the baseline SLD. The baseline SLD will |
| | be used as the reference by which the objective tumor |
| | response or progression will be characterized. |
| | All other lesions (or sites of disease) should be identified |
| | as non-target lesions and should be recorded at baseline. |
| | Measurements of these lesions are not required, but the |
| | presence or absence of each should be noted throughout |
| | follow-up. The bone windows of the CT scans must be |
| | reviewed to identify bone metastases. |
| 13. | Eligibility checklist. Inclusion and Exclusion criteria. |

### Pre-Cycle 1 for Subjects on Picoplatin Arm

These evaluations must be completed on Day 1 of Cycle 1 prior to treatment:
1. Weight.
2. Vital signs. Vital signs will be measured again at the end of the infusion.
3. ECOG PS
   If the following evaluations were completed for the screening process within 3 days of Cycle 1 treatment, they did not need to be repeated.
1. Abbreviated physical examination. This consists of examination of the neck, chest, abdomen, and any abnormal areas indicated by previous exam, interval symptoms, signs, laboratory or radiographic data.
2. White blood cell count (WBC) with differential, Hgb, and platelet count.
3. Serum chemistry: BUN, creatinine, glucose, sodium, potassium, chloride, bicarbonate, calcium, phosphorus, magnesium uric acid, total bilirubin, alkaline phosphatase, LDH, SGOT/AST, SGPT/ALT, albumin, total protein.

### On-Therapy Evaluation for Subjects on Picoplatin Arm

Mid-Cycle of Cycles 1, 2, and 3 and any Cycle which Required Dose Reduction

White blood cell count (WBC) with differential, Hgb, and platelet counts was to be obtained once between Days 11-15 of Cycles 1, 2, and 3 and during any cycle in which a dose reduction was required for hematological toxicity.

If the platelet count is <100×10⁹/L or the ANC is <1.5×10⁹/L, then laboratory tests should have been repeated at a minimum of weekly intervals to see if the required laboratory values are reached. In the event of an ANC <0.5×10⁹/L or a platelet count <25×10⁹/L, hematology values were to be monitored at least three times a week until the neutrophil and platelet counts have risen above these levels.

Prior to Each Treatment Cycle from Cycle 2

The evaluations on Table 17 were to take place every 3 weeks. If treatment was delayed because of toxicity, the evaluations were to be done as originally scheduled, i.e., 3 weeks after the last dose of picoplatin. Items 1-7 were then to be repeated at least weekly, including just prior to the next dose of picoplatin. For serum chemistries, only those that were abnormal needed be repeated.

TABLE 17
Evaluations During Treatment

| | |
|---|---|
| 1. | Adverse event query. |
| 2. | Abbreviated physical exam. This consists of examination of |
| | the neck, chest, abdomen, peripheral nerves, and any |
| | abnormal areas indicated by previous exam, interval |
| | symptoms, signs, laboratory or radiographic data. |
| 3. | Vital signs including pulse, blood pressure |
| | (systolic/diastolic), respiratory rate, and temperature. |
| 4. | Weight. If a weight change of 10% or more has occurred, |
| | recalculate BSA for study drug dose. |
| 5. | ECOG PS. |
| 6. | Concomitant medication query. |
| 7. | White blood cell count (WBC) with differential, Hgb, and |
| | platelet count. |
| 8. | Serum chemistry: BUN, creatinine, glucose, sodium, |
| | potassium, chloride, bicarbonate, calcium, phosphorus, uric |
| | acid, total bilirubin, alkaline phosphatase, LDH, |
| | SGOT/AST, SGPT/ALT, albumin, and total protein. |
| 9. | Calculated creatinine clearance. |
| 10. | During Cycles 1, 2, and 3, and thereafter during any cycle |
| | for which the dose of chemotherapy was reduced because |
| | of hematological toxicity: WBC with differential, Hgb, and |
| | platelet count once between Days 11-15. |
| 11. | Pregnancy tests for women of child-bearing potential, as |
| | required, per regional practice. |

### At the End of Every Second Treatment Cycle, (Prior to Cycles 3, 5, 7, Etc.)

TABLE 18
Evaluations During Treatment

| | |
|---|---|
| 1. | Urinalysis: dipstick, with microscopic examination of |
| | sediment in the event of an abnormal reading on the |
| | dipstick. |
| 2. | CT chest and abdomen, as performed at baseline. |
| | Evaluation of all known tumor sites present at baseline |
| | (including brain) should be performed prior to every other |
| | chemotherapy cycle (approximately every 6 weeks) while |
| | subjects continue to receive picoplatin. Both measurable |
| | and non-measurable lesions must be assessed for response |
| | or progression and new lesions must be documented. |
| | Target lesions must be measured and recorded to document |
| | a response as a complete response (CR) or partial response |
| | (PR) or whether the disease is stable (SD) or has progressed |
| | (PD). Assessments of tumor status and lesions should |
| | utilize the same methods used for the baseline assessments. |
| | The radiological determination of disease response or |
| | progression will be made by either an investigator and a |
| | radiologist, or 2 radiologists reviewing the CT scans and |
| | using RECIST. |
| 3. | Additional diagnostic procedures as needed to assess fully |
| | any new symptoms or signs that might indicate PD (e.g., |
| | bone scan). |

Evaluations for Subjects on BSC Alone Arm and for Subjects Who have Discontinued Picoplatin for any Reason

TABLE 19
Evaluations Every 3 Weeks

1. Abbreviated physical exam included examination of the
chest, neck, abdomen, peripheral nerves and any system
abnormal at baseline.
2. Interval medical history for AE query.
3. Vital signs including pulse, blood pressure
(systolic/diastolic), respiratory rate, and temperature.
4. ECOG PS.
5. Concomitant medication query.
6. Weight.
7. White blood cell count (WBC) with differential, Hgb, and
platelet count.
8. Serum chemistry: BUN, creatinine, glucose, sodium,
potassium, chloride, bicarbonate, calcium, phosphorus, uric
acid, total bilirubin, alkaline phosphatase, LDH, SGOT/AST,
SGPT/ALT, albumin and total protein.

### Evaluations Every 6 Weeks Until Documented Progression

TABLE 20
Six Week Evaluations

| | |
|---|---|
| 1. | CT chest and abdomen (including entire liver and adrenal glands). |
| | Evaluation of all known tumor sites present at baseline (including |
| | brain) was to be performed every 6 weeks until progression was |
| | documented. Both measurable and non-measurable lesions were to be |
| | assessed for response or progression and new lesions must be |
| | documented. Target lesions must be measured and recorded to |
| | document CR, PR, SD, or PD. Assessments of tumor status and |
| | lesions should have utilized the same methods used for the baseline |
| | assessments. |
| 2. | Additional diagnostic procedures as needed to assess fully any new |
| | symptoms or signs that might indicate PD (e.g., bone scan). |

### Survival Follow-Up

If a subject decided to discontinue participation in the study, was unable to comply with the protocol, or if a subject started a further chemotherapy regimen, they were considered in survival follow-up. They were to be contacted every month by telephone, unless consent for survival follow-up was specifically withdrawn. During survival follow-up, information was to be collected on any anti-cancer therapy the subject may be receiving and the survival status of the subject was to be documented. Physical exams, lab evaluations, adverse events, concomitant medications and medical procedures were not to be collected on the CRFs, and no further CT scans are required.

### Definition of Endpoints and Criteria for Evaluation Primary Endpoint

Overall survival: was measured from the date of randomization to the date of death from any cause. For each subject who was not known to have died, overall survival duration will be censored at the date the patient was last known to be alive.

### Secondary Endpoints

Proportion of subjects with an objective response: was measured as the proportion of subjects who achieved radiological evidence of a CR or PR. For this analysis all subjects in the analysis population, who did not meet the criteria as specified by RECIST for a CR or PR will be included as if they did not have a response. The categorization of response used the best overall response recorded from the initiation of study drug. Objective response required a confirmatory exam documenting the response at least 4 weeks later.

Proportion of subjects with disease control: was measured as the proportion of subjects who achieved radiological evidence of a CR, PR, or SD. For this analysis, all subjects in the analysis population, who did not meet the criteria as specified by RECIST for a CR, PR, or SD were included as if they have progressed. Complete response and PR required a confirmatory exam documenting the response at least 4 weeks later. Stable disease was documented by a follow-up CT scan after study entry at a minimum interval of not less than 6 weeks; this did not require a confirmatory exam.

Duration of response: was measured from the date that the subjects first meets the criteria of response to the date that the subjects progressed (radiologically or symptomatically), or until death from any cause. For each subject that was not known to have progressed or died, duration of response was censored at the date that the subjects was last known to be alive and progression-free.

Progression-free survival: was measured from the date of randomization to the date that the subjects progressed (radiologically or symptomatically), or until death from any cause. For each subject that was not known to have progressed or died, PFS was censored at the date that the subjects was last known to be alive and progression free.

Determination of radiographic disease status for continuation of study drug and analyses of response or progression were determined by the investigator(s) and/or local radiologist(s) using RECIST.

### Safety

### Adverse Events (AE)

The definition of an AE followed the ICH E6: GCP Step 5, Consolidated Guideline 1.5.96, April 1998 edition:

An AE is any untoward medical occurrence which occurred after randomization, and which did not necessarily have a causal relationship with the study drug. An AE can therefore be any unfavorable and unintended sign (including an abnormal laboratory finding), symptom, or disease temporally associated with the use of the study drug, whether or not related to the study drug.

An unexpected AE is any AE that occurred following the administration of study drug for which the specificity or the severity is not consistent with the current Investigator's Brochure.

A laboratory abnormality was to be reported as an AE only if it was associated with clinical sequelae or requires a therapeutic intervention, including a delay in the administration of picoplatin or a reduction in the dose of picoplatin administered.

Investigators or their designees were to follow subjects for AEs from randomization until death, discontinuation from study or until the end of the study. Safety of picoplatin was assessed by comparing the frequency, severity and nature of the AEs between the safety populations of the 2 treatment arms during the “drug safety period.”

### Grading and Relationship to Study Drug

Adverse events that occurred during the study will be graded using the NCI CTCAE v3.0. Adverse events not included in the CTCAE were to be graded according to the following table.

TABLE 21
Toxicity Grading for Adverse Events not included in the CTCAE

| Grade/Severity | Description |
|---|---|
| GRADE 1—Mild | Transient or mild discomfort; no limitation in activity; |
| | no medical intervention/therapy required. |
| GRADE | Mild to moderate limitation in activity - some |
| 2—Moderate | assistance may be needed; no or minimal medical |
| | intervention/therapy required. |
| GRADE | Marked limitation in activity, some assistance usually |
| 3—Severe | required; medical intervention/therapy required, |
| | hospitalizations possible. |
| GRADE 4—Life | Extreme limitation in activity, significant assistance |
| Threatening | required; life threatening (immediate risk of death); |
| | significant medical intervention/therapy required, |
| | hospitalization or hospice care probable. |
| GRADE 5—Fatal | Death |

Association or relatedness to test agent will be identified as follows:
Not Related: Exposure to the investigational product did not occur, or the occurrence of the AE is not reasonably related in time, or the event is readily and more likely explained by the patient's clinical state or by other modes of therapy administered to the patient.
Possibly Related: Follows a reasonable temporal sequence from drug administration; but could have been produced by the patient's clinical state or by other modes of therapy administered to the patient.
Probably Related: The study treatment and the AE were reasonably related in time, and the AE is more likely explained by exposure to the study product than by other causes, or the investigational product was the most likely cause of the AE.

### Serious Adverse Events (SAE)

An SAE is defined as any AE that results in any of outcomes, listed on Table 22 below.

TABLE 22
Serious SAE

1. Death.
2. A life-threatening adverse experience.
3. Inpatient hospitalization or prolongation of an existing hospitalization.
4. A persistent or significant disability/incapacity.
5. A congenital anomaly/birth defect.
6. An important medical event that may not result in death, be life
threatening, or require hospitalization may be considered a SAE, when,
based upon appropriate medical judgment, it may jeopardize the subject
and may require medical or surgical intervention to prevent one of the
outcomes listed in this definition.

### Statistical and Analytical Plans

### Handling of Missing and Incomplete Data

Missing data will be queried. If the data cannot be obtained, conventions for missing data will be pre-specified for all data not used in the analyses of the primary or secondary endpoints. Data will not be imputed for any missing primary or secondary endpoint variables.

Incomplete data resulting from subjects who terminate the study for reasons other than disease progression or death will be censored for the time-to-event analyses. Investigators must use every possible reasonable effort to obtain survival information on all subjects regardless of the reasons for discontinuation from the study. For each subject that is lost to follow-up or not known to have died at the time of data cutoff, overall survival duration will be censored at the date that he/she was last known to be alive.

### Populations for Analysis

Intent-to-Treat (ITT) Population included all randomized subjects according to the treatment group to which they were randomized, regardless of whether or not the subject received any study drug.

This population was used for all the efficacy endpoints.

Radiographically-Evaluable (RE) Population included all randomized subjects meeting the following criteria: (a) had measurable disease (using RECIST) at baseline; and (b) had at least one evaluable post-baseline tumor assessment. This population will be used for the secondary response endpoints other than PFS.

### Efficacy Analysis

Tabular results will be displayed primarily by treatment group. There may be some tables where information is also displayed by region, cycle, or stratification factor(s) within treatment group. Kaplan Meier plots will be prepared for time-to-event analyses.

### Primary Endpoint

The primary endpoint is overall survival. The primary analysis of this endpoint was performed on the ITT population. Overall survival was summarized by treatment group using Kaplan Meier methods.

The primary test for differences in overall survival between the 2 treatment arms was evaluated using a 2-sided stratified log-rank test with α=0.05 significance level adjusted for the interim analyses. The test was stratified according to the specified stratification factors: (a) geographic region (Europe vs. India vs. South America); (b) response to prior therapy (refractory vs. progressed within 91 to 180 days); (c) ECOG PS at baseline (0 or 1 vs. 2); and/or (d) ≦50 years of age. Performance of subsets patients who (a) did not respond to first-line therapy, (b) patients who did not response to first-line therapy or responded to first-line therapy and then relapsed within 45 days and (c) patients who did not receive post-study (third-line) chemotherapy were also evaluated.

Additional supportive analyses will include log-rank tests of the stratification factors and Cox proportional hazards regression analysis of overall survival to investigate the effect of the stratification factors on survival. Other prognostic factors may be investigated in an exploratory nature, as deemed appropriate. All supportive analyses will be evaluated using a 2-sided test with α=0.05 significance level.

### Secondary Endpoints

The analyses of the response endpoints will use the determination of disease response or progression as determined by the investigator and/or local radiologist using RECIST.

The proportion of subjects who achieve an objective response, and the proportion of subjects who achieve disease control will be assessed in the ITT and RE populations. These endpoints will be presented by treatment arm along with 95% confidence intervals. Each proportion will be compared between treatment arms using a Fisher's exact test.

Logistic regression will be used to investigate the effect of the stratification factors on each of these endpoints. Other prognostic factors may be investigated in an exploratory nature, as deemed appropriate.

Duration of response will be assessed in the ITT and RE populations and PFS will be assessed in the ITT population. These time-to-event endpoints will be assessed using the Kaplan-Meier method and the difference between the two treatment arms will be compared using a stratified log-rank test.

All statistical tests will be evaluated with a 2-sided α=0.05.

### Safety Analysis

Safety will primarily be assessed by comparing the frequency, severity, and nature of the AEs between the safety populations of the two treatment arms during the “drug safety period”. Any comparison against baseline will use the measurement immediately preceding the start of study medication (for the picoplatin subjects) or immediately preceding to the date of randomization (for BSC subjects).

Due to inherent differences in the treatment arms (i.e., the BSC arm will not have a first or last dose of study drug) a “drug safety period” has been defined to allow for the evaluation of drug safety over a time period that will be long enough to assess safety and permit reasonable comparisons between the treatment arms. The “drug safety period” is defined as the time from the date of randomization to 30 days after the last date of study drug administration; 7 days after documentation of progressive disease; or Study Week 12 (Day 84), whichever occurs later, unless the subject discontinues from the study prior to this time.

Adverse events will be summarized by preferred term and body system for each treatment group, cycle, and visit. The MedDRA AE dictionary will be used to map all verbatim AEs to preferred terms and body systems. The frequency of AEs experienced by all subjects will be computed by the treatment cycle of event onset and overall for each treatment group. The tabulation will count the number of subjects reporting each preferred term and the total number of subjects reporting at least 1 event per body system. Summary tables will tabulate AEs by relatedness to study drug, maximum severity (toxicity) and treatment cycle of AE onset. In the event that a subject experiences multiple occurrences of the same event, then the event “most related” to study drug or most severe will be used in the analyses.

Serious adverse events, AEs leading to study drug discontinuation, and laboratory AEs will be summarized by subject listing and described by narrative, where appropriate.

### Concomitant Medications

Concomitant medication use will be summarized for the safety population by treatment group. This summary will include all medications taken during the drug safety period. The WHODRUG medication dictionary will be used for coding medications.

### Exposure to Study Drug

Study drug exposure will be summarized by treatment cycle and overall. The initiation of study drug will be considered Day 1 of Cycle 1. The day of administration during the next cycle of study drug will be considered Day 1 of Cycle 2, etc. The drug exposure information will include dosing information, length of cycles, and dose intensity. The number of dose delays and dose reductions will be summarized. Descriptive statistics will be used to summarize this information.

### ECOG Performance Status

Performance status will be assessed and summarized by comparing baseline values to each scheduled visit. Values will be categorized as improved, maintained, or worsened and will be presented using frequency statistics.

### Results

Patients received either picoplatin with BSC (n=268) or BSC alone (n=133). In each arm of the trial, about 85% of the patients enrolled had an ECOG PS of 0-1 and about 15% had an ECOG of 2. The number of cycles (treatments) with picoplatin ranged from 1-15 with a median of 3, at a median cycle length (time between treatments) of 21-42 days (median=22). The median dose intensity (mg/m²/21 days) was 120 mg/m². Post-study chemotherapy was administered to 40% of the BSC-only patients and to 27% of the patients who received BSC plus picoplatin. After progression, 28% vs. 41% of patients received post-study chemotherapy for picoplatin vs. BSC. The most common post-study chemotherapy regimens included CAV, topotecan, paclitaxel, or carboplatin/cisplatin (alone or in combinations).

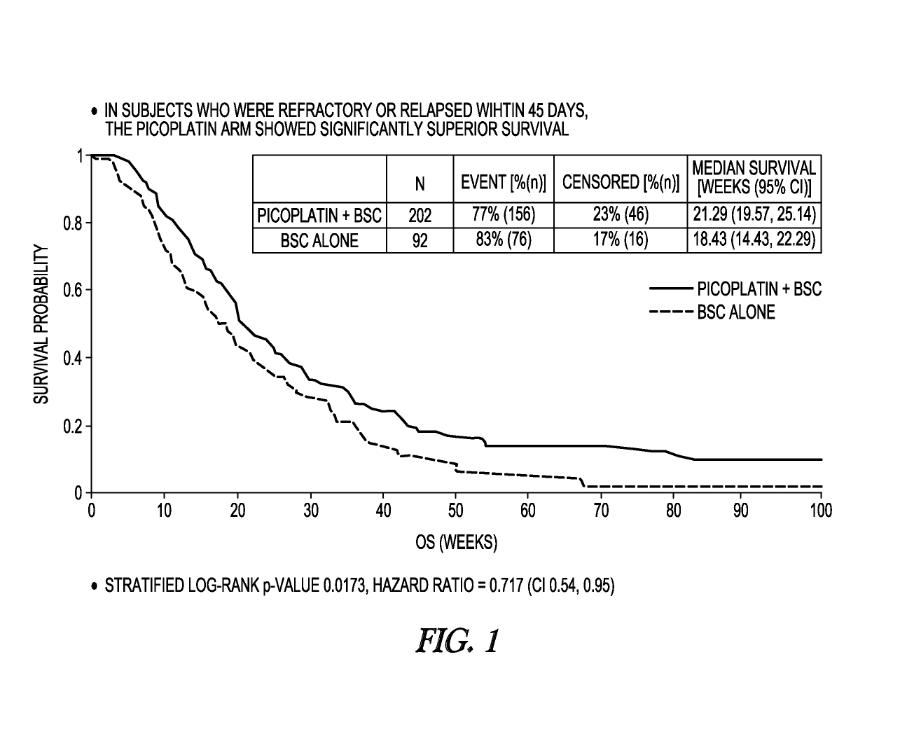

Disease control (PR+CR+SD) was observed in 48.2% of the picoplatin plus BSC group vs. 27.4% of the BSC-only group. With a hazard ratio (HR) of 0.8, median survival time (MST) for picoplatin vs BSC was 20.57 wks (CI 19-25) vs 19.71 wks (CI 16-24) (p=0.09). However, those refractory patients who never responded or patients who relapsed within 45 days had a significant improvement in MST (2.86 weeks) when treated with picoplatin and BSC vs BSC. The median survival for these patients treated with picoplatin plus BSC was 21.29 wks vs 18.43 wks for BSC-only patients (see FIG. 1). Those highly refractory patients who did not respond to first-line therapy achieved a survival benefit of 2.14 weeks if treated with picoplatin plus BSC, that increased to over 4 weeks if they entered the trial with an ECOG PS of 0 or 1.

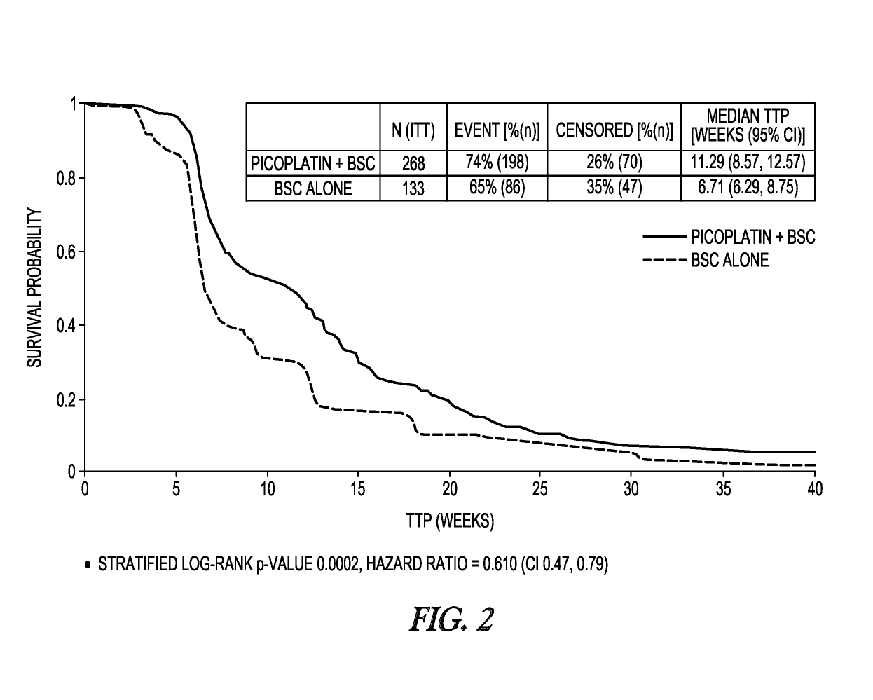

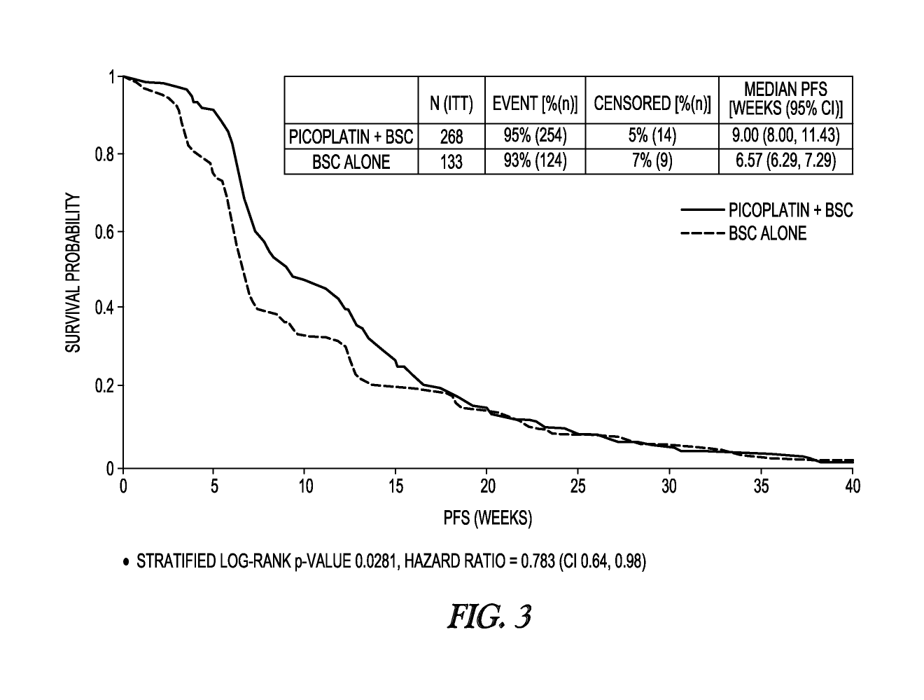

The medium TTP for the patients receiving picoplatin plus BSC was 11.29 wks vs 6.71 wks for patients receiving BSC (see FIG. 2). PFS for the patients receiving picoplatin plus BSC was 9.00 wks vs 6.57 wks for patients receiving BSC (FIG. 3). Thus, these surrogate endpoints of disease control, showed statistical significance in favor of picoplatin, although the primary endpoint of OS was not met.

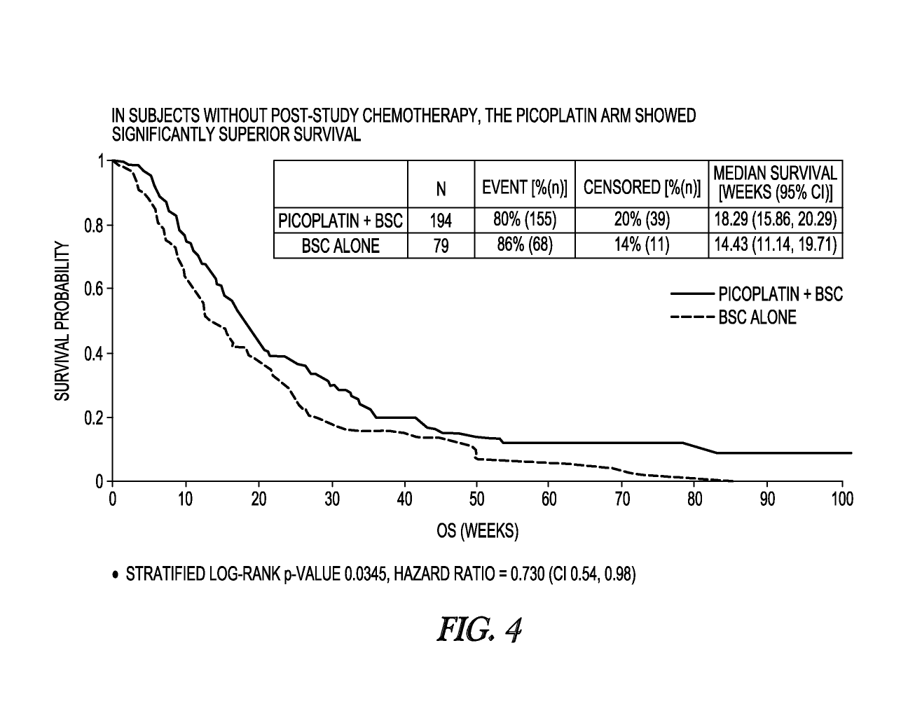

However, after progression, 28% vs 41% of patients received post study chemotherapy for picoplatin plus BSC treatment vs BSC only. In patients who did not receive post study chemotherapy, MST for picoplatin (n=194) was 18 wks (CI 16-20) and for BSC (n=79) was 14 wks (CI 11-20) (FIG. 4). Therefore, post-study chemotherapy may have been a contributing factor affecting the trial outcome.

Adverse events (AEs) for BSC patients were associated with SCLC. On picoplatin, grade 3/4 AEs >10% were thrombocytopenia (44%), anemia (29%), neutropenia (18%), asthenia (11%). Febrile neutropenia occurred in 2 patients (<1%). No hypersensitivity or picoplatin treatment-related deaths due to hematologic toxicity were observed.

Adverse events related to picoplatin administration are summarized on Table 23, below.

TABLE 23

| | | |
|---|---|---|
| All Grades | Grade 3 | Grade 4 |

Adverse Events Related to Picoplatin in >5% of patients (n = 266)

| | | | | | | |
|---|---|---|---|---|---|---|
| Hematological | 220 | (83%) | 83 | (31%) | 74 | (28%) |
| Thrombocytopenia | 185 | (70%) | 59 | (22%) | 57 | (21%) |
| Bleeding* | 11 | (4%) | 0 | | 0 | |
| Anemia | 148 | (56%) | 60 | (23%) | 17 | (6%) |
| Neutropenia** | 100 | (38%) | 36 | (14%) | 13 | (5%) |
| Nausea | 24 | (9%) | 0 | | 0 | |
| Vomiting | 18 | (7%) | 1 | (0.4%) | 0 | |
| Asthenia | 15 | (6%) | 0 | | 0 | |

Adverse Events of Special Interest Related to Picoplatin

| | | | | | |
|---|---|---|---|---|---|
| Peripheral neuropathy | 10 | (4%) | 2 | (0.8%) | 0 |
| Decreased creatinine | 12 | (5%) | 0 | | 0 |
| clearance | | | | | |

*Bleeding events were mild, Grade 1-2 hemoptysis, epitaxis, petechiae, purpura and hematuria
**Febrile neutropenia was observed in only 2 patients

Of 266 treated patients only 13 (4.9%) discontinued picoplatin due to adverse events; 10 (3.8%) due to thrombocytopenia; 3 (1.1%) due to anemia; 1 (0.4%) due to pancytopenia, 1 (0.4%) due to increased LDH; and 1 (0.4%) due to encephalopathy.

### Example 3

### Treatment of SCLC Brain Metastases

### Intracranial Orthotopic SCLC Model

Picoplatin efficacy was assessed in an orthotopic model of SCLC brain metastasis by Charles River Laboratories, Discovery and Imaging Services, previously known as MIR Preclinical Services, Ann Arbor, Mich. 10⁶ DMS 114 ACLC cells were implanted intracranially in 9-10 week-old female athymic nude mice (outbred nu/nu). Treatment with vehicle or picoplatin (35 mg/kg) began on day 18 at a mean tumor weight of 13 mg with a dosing schedule of Q7DX4. Magnetic resonance imaging (MRI) was used to assess tumor mass and doubling time (Td) twice a week. [¹⁸F] fluorothymidine positron emission tomography (FLT PET) was used as an indicator of tumor proliferative index, expressed as the standardized uptake value (SUV), calculated as follows: SUV=(Mean tumor ¹⁸F activity (μCi/g)×body weight (g))÷decay-adjusted injected dose (μCi).

An initial time course for PET (24, 48, and 72 h) was performed to determine optimal post-treatment imaging time, and subsequent post-dose PET imaging was performed at the optimal time (24 h). All procedures carried out in this experiment were conducted in compliance with all the laws, regulations and guidelines of the National Institutes of Health (NIH) and with the approval of MIR's Animal Care and Use Committee. Statistical pairwise comparisons of the MRI tumor volume and PET SUV data were performed using the Wilcoxon rank sum test. P values were adjusted using the method of Holm (non-parametric).

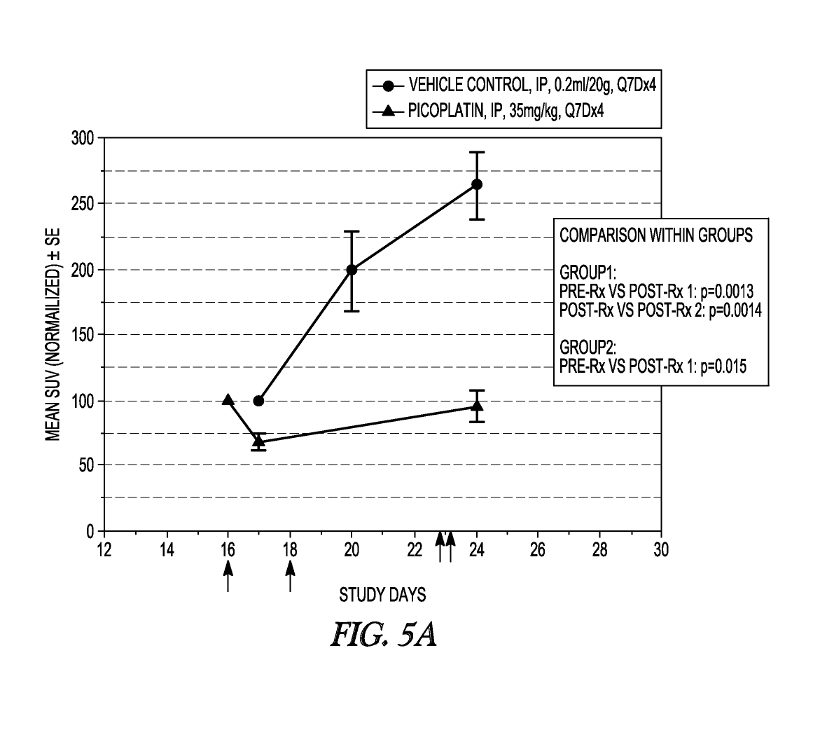

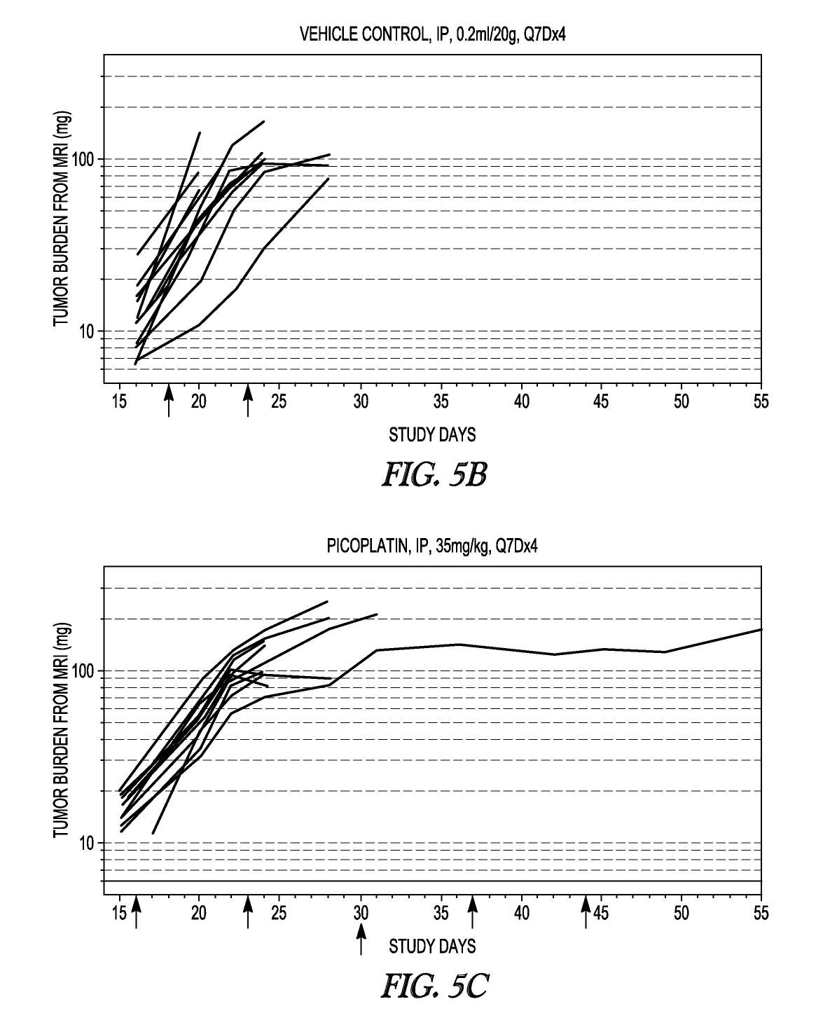

In this model of SCLC brain metastasis, a decrease in cell proliferation, an increase in tumor doubling time, and in one animal treatment-related tumor stasis and increase in survival time was observed. Although the observed trend toward reduced tumor burden in the picoplatin treatment group did not reach statistical significance, technical factors may have contributed to the observation of a trend, rather than statistical significance. For logistical reasons, initial MRI tumor volume measurements for the control group were taken a day before that of the picoplatin (24 h) group and animals were subsequently triaged for equal mean tumor volume. It is therefore likely that tumors in the control group were actually larger than those of the picoplatin group at the same timepoint. See FIG. 5.

In addition, four mice in the control group were lost after MRI. Three of the four animals lost contained the three largest tumor volumes in the control group, which reduced the mean tumor volume of the control group. In light of these factors, it is believed that observation of picoplatin anti-tumor activity in SCLC growing in the brain is significant.

### REFERENCES

All references below, and all other documents, patents, and publications referred to herein are incorporated by reference in their entireties.
Jemal A, Tiwari Ra, Murray T, et al. Cancer Statistics, 2004. CA Cancer J Clin 2004; 54(1):8-29.
Vaporciyan A A, Kies M, Stevens C, et al. Cancer of the Lung; The Thorax. In: Cancer Medicine. 6 ed; 2003. p. 1385-1445.
Clark R, Ihde D C. Small-cell lung cancer: treatment progress and prospects. Oncology (Williston Park) 1998; 12(5):647-58; discussion 661-3.
Glisson B S. Recurrent small cell lung cancer: update. Semin Oncol 2003; 30(1):72-8.

Davies A, Evans W, Mackay J, et al. Treatment of recurrent small cell lung cancer. Hematology/Oncology Clinics of North America 2004; 18:387-416.
Murray N. Second-Line Chemotherapy for Small-Cell Lung Cancer. In: American Society of Clinical Oncology, Education Handbook; 2003. p. 667-71.
Jackman D M, Johnson B E. Small-cell lung cancer. Lancet 2005; 366(9494):1385-96.
Sundstrom S, Bremnes R M, Kaasa S, et al. Second-line chemotherapy in recurrent small cell lung cancer. Results from a crossover schedule after primary treatment with cisplatin and etoposide (EP-regimen) or cyclophosphamide, epirubicin, and vincristin (CEV-regimen). Lung Cancer 2005; 48(2):251-61.
National Comprehensive Cancer Network. Clinical Practice Guidelines in Oncology, Small Cell Lung Cancer, Principles of Supportive Care. 2006; Version 1:p. SCL-B.
Huisman C, Postmus P E, Giaccone G, et al. Second-line chemotherapy and its evaluation in small cell lung cancer. Cancer Treat Rev 1999; 25(4):199-206.
Giaccone G, Donadio M, Bonardi G, et al. Teniposide in the treatment of small-cell lung cancer: the influence of prior chemotherapy. J Clin Oncol 1988; 6(8):1264-70.
Raynaud F I, Boxall F E, Goddard P M, et al. cis-Amminedichloro(2-methylpyridine) platinum(II) (AMD473), a novel sterically hindered platinum complex: in vivo activity, toxicology, and pharmacokinetics in mice. Clin Cancer Res 1997; 3(11):2063-74.
Holford J, Raynaud F, Murrer B A, et al. Chemical, biochemical and pharmacological activity of the novel sterically hindered platinum co-ordination complex, cis-[amminedichloro(2-methylpyridine)] platinum(II) (AMD473). Anticancer Drug Des 1998; 13(1):1-18.
Holford J, Sharp S Y, Murrer B A, et al. In vitro circumvention of cisplatin resistance by the novel sterically hindered platinum complex AMD473. Br J Cancer 1998; 77(3):366-73.
Rogers P, Boxall F E, Allott C P, et al. Sequence-dependent synergism between the new generation platinum agent ZD0473 and paclitaxel in cisplatin-sensitive and -resistant human ovarian carcinoma cell lines. Eur J Cancer 2002; 38(12):1653-60.
Sharp S Y, O'Neill C F, Rogers P, et al. Retention of activity by the new generation platinum agent AMD0473 in four human tumour cell lines possessing acquired resistance to oxaliplatin. Eur J Cancer 2002; 38(17):2309-15.
Beale P, Judson I, O'Donnell A, Trigo J, Rees C, Raynaud F, A Phase I clinical and pharmacological study of cis-diamminedichloro(2-methylpyridine) platinum II (AMD473). British Journal of Cancer 2003; 7(88):1128-1134.
Treat J, Schiller J, Quoix E, et al. ZD0473 treatment in lung cancer: an overview of the clinical trial results. European Journal of Cancer 2002; 38(8):S13-S18.
Giaccone G, O'Brien M E, Byrne M J, et al. Phase II trial of ZD0473 as second-line therapy in mesothelioma. Eur J Cancer 2002; 38 Suppl 8:S19-24.
Gore M E, Atkinson R J, Thomas H, et al. A Phase II trial of ZD0473 in platinum-pretreated ovarian cancer. Eur J Cancer 2002; 38(18):2416-20.
Ebi N, Kubota K, Nishiwaki Y, et al. Second-line chemotherapy for relapsed small cell lung cancer. Jpn J Clin Oncol 1997; 27(3):166-9.
Ardizzoni A, Hansen H, Dombernowsky P, et al. Topotecan, a new active drug in the second-line treatment of small-cell lung cancer: a Phase II study in patients with refractory and sensitive disease. The European Organization for Research and Treatment of Cancer Early Clinical Studies Group and New Drug Development Office, and the Lung Cancer Cooperative Group. J Clin Oncol 1997; 15(5):2090-6.
Gelmon, K. A., et al., A Phase 1 Study of AMD473 and Docetaxel Given Once Every Three Weeks In Patients with Advanced Refractory Cancer. Annals of Oncology 2004, 15, 1115-1124.
von Pawel J, Schiller J H, Shepherd F A, et al. Topotecan versus cyclophosphamide, doxorubicin, and vincristine for the treatment of recurrent small-cell lung cancer. J Clin Oncol 1999; 17(2):658-67.
Ardizzoni A. Topotecan in the treatment of recurrent small cell lung cancer: an update. Oncologist 2004; 9 Suppl 6:4-13.
Perez R P. Cellular and molecular determinants of cisplatin resistance. Eur J Cancer 1998; 34(10):1535-42.
Markman M. Topotecan as second-line therapy for ovarian cancer: dosage versus toxicity. Oncologist 2005; 10(9):695-7.
O'Brien M E R, Ciuleanu T, Tsekov H, et al. in relapsed, resistant SCLC, oral topotecan plus supportive care offers survival benefit compared to supportive care alone. Proceedings from the 2005 Annual Chemotherapy Foundation Symposium 2005.
de Wet M, Falkson G, Rapoport B L. Small cell lung cancer: analysis of factors influencing the response to treatment and survival. Oncology 1994; 51(6):523-34.
Paesmans M, Sculier J P, Lecomte J, et al. Prognostic factors for patients with small cell lung carcinoma: analysis of a series of 763 patients included in 4 consecutive prospective trials with a minimum follow-up of 5 years. Cancer 2000; 89(3):523-33.
Bremnes R M, Sundstrom S, Aasebo U, et al. The value of prognostic factors in small cell lung cancer: results from a randomised multicenter study with minimum 5 year follow-up. Lung Cancer 2003; 39(3):303-13.
National Comprehensive Cancer Network. Small Cell Lung Cancer Guideline, Version 1 (2008). Available at: http://www.nccn.org/professionals/physician_gls/pdf/scic.pdf;
National Comprehensive Cancer Network. Palliative Care Guideline, Version 1 (2007). Available at: http://www.nccn.org/professionals/physician_gls/PDF/palliative.pdf
Application No. PCT/US2008/001746, filed Feb. 8, 2008, published as WO2008/097658, entitled “Encapsulated Picoplatin”;
Application No. PCT/US2008/001752, filed Feb. 8, 2008, published as WO2008/097661, entitled “Stabilized Picoplatin Oral Dosage Form”;
Application No. PCT/US2008/008669, filed Jul. 16, 2008, published as WO 2009/011861, entitled “Oral Formulations for Picoplatin”;
Application No. PCT/US2008/008076, filed Jun. 27, 2008, published as WO2009/032034, entitled “Stabilized Picoplatin Dosage Form”;
Application No. PCT/US2009/000770, filed Feb. 6, 2009, published as WO2009/099649, entitled “Use of Picoplatin and Bevacizumab to Treat Colorectal Cancer”;
Application No. PCT/US2009/000773, filed Feb. 6, 2009, published as WO2009/099651 entitled “Use of Picoplatin and Cetuximab to Treat Colorectal Cancer”;
Application No. PCT/US2009/000750, filed Feb. 6, 2009, published as WO2009/099634 entitled “Picoplatin and Amrubicin to Treat Lung Cancer”;
Application No. PCT/US2010/00735, filed Mar. 11, 2010. U.S. Ser. No. 10/276,503, filed Sep. 4, 2003, entitled “Combination Chemotherapy”;
U.S. Ser. No. 11/982,839, filed Nov. 5, 2007, entitled “Use of Picoplatin to Treat Small Cell Lung Cancer”;
U.S. Ser. No. 11/982,841, filed Nov. 5, 2007, entitled “Use of Picoplatin to Treat Colorectal Cancer”;
U.S. Ser. No. 11/935,979, filed Nov. 6, 2007, entitled “Use of Picoplatin to Treat Prostate Cancer”;
U.S. Ser. No. 12/367,394, filed Feb. 6, 2009, “Use of Picoplatin to Treat Colorectal Cancer”;
U.S. Ser. No. 12/464,662, filed May 12, 2009, “Use of Picoplatin to Treat Colorectal Cancer”;
U.S. Ser. No. 12/465,563, filed May 13, 2009, “Use of Picoplatin to Treat Colorectal Cancer”;
U.S. Ser. No. 12/635,534, filed Dec. 10, 2009, “Combination Therapy for Ovarian Cancer”;

all of which are incorporated by reference herein in their entireties.

1. A method of treating small cell lung cancer (SCLC) comprising:
(a) selecting a patient who was non-responsive to initial therapy employing a drug that is inactivated by a mechanism comprising intracellular glutathione binding or who responded to said therapy but then relapsed within 45 days of cessation of said initial therapy; and
(b) administering to said non-responsive or relapsed patient an amount of picoplatin and providing a regimen of best supportive care (BSC) during the period in which further therapy with said drug is less effective, wherein the picoplatin is effective to extend progression-free survival or the life expectancy of the patient.

2. The method of claim 1 wherein the patient responded to initial therapy but then relapsed within 45 days of cessation of said therapy.

3. The method of claim 1 wherein the patient is non-responsive to said initial therapy.

4. The method of claim 1 wherein the drug is cisplatin and/or carboplatin.

5. The method of claim 1 wherein said SCLC is progressive after two or more cycles of said therapy.

6. The method of claim 1 wherein the SCLC is stable after three cycles of said therapy.

7. The method of claim 1 wherein picoplatin and a additional chemotherapeutic agent is administered during step (b).

8. The method of claim 1 wherein a regimen of BSC is provided to the patient during step (b) and further comprising step (c) wherein said SCLC progresses and said patient is not treated for a period of time with subsequent therapy with a platinum- or non-platinum containing anti-cancer agent.

9. The method of claim 8 wherein the patient does not receive said subsequent therapy within about 60 days of said progression.

10. The method of claim 1 wherein the SCLC patient is afflicted with brain metastases that are treated with said picoplatin.

11. The method of claim 1 wherein said selected patient has an ECOG performance score of 0 or 1.

12. The method of claim 1 comprising administering at least two cycles of picoplatin in step (b).

13. A method to extend the period of effective treatment of SCLC employing the administration of a Pt-containing anti-cancer agent comprising:
(a) selecting a patient afflicted with SCLC who has not responded to a platinum-containing anti-cancer drug comprising cisplatin and/or carboplatin wherein said patient has remained stable after at least 3 cycles of the drug or progressed through at least two cycles of the drug, or who has responded to said drug but then relapsed within 45 days of receiving said drug; and
(b) administering an effective amount of picoplatin to said patient during a period, wherein the life of the SCLC patient is extended over that of a SCLC patient afflicted with SCLC that has not responded to said anti-cancer drug or that has responded then relapsed within 45 days from cessation of treatment with said drug and who then does not receive picoplatin but receives BSC during the period.

14. The method of claim 12 wherein the SCLC patient of step (a) has an ECOG performance score of 0 or 1.

15. The method of claim 12 wherein the SCLC patient of step (a) is afflicted with brain metastases that are treated by the picoplatin.

16. A method of treating small cell lung cancer (SCLC) comprising:
(a) selecting a human patient afflicted with SCLC, wherein the patient was non-responsive to first-line therapy comprising carboplatin or cisplatin, or wherein the SCLC responded to said therapy but relapsed within about 180 days of cessation of said therapy;
(b) administering an effective amount of picoplatin and a regimen of best supportive care (BSC) to said patient,
wherein the lifespan and/or progression free survival (PFS) of the patient administered the picoplatin are extended over that of a patient selected in step (a) who receives only a regimen of BSC, wherein the picoplatin-treated patient does not receive third-line chemotherapy, following progression of the SCLC, for a period of time.

17. The method of claim 16 wherein the patient administered picoplatin also receives BSC during picoplatin administration, and after picoplatin administration.

18. The method of claim 16 wherein the period of time is up to about one year from progression of the SCLC.

19. The method of claim 18 wherein the period of time is up to about 60 days from progression of the SCLC.

20. The method of claim 16 wherein the SCLC relapsed within about 45 or 90 days of cessation of first-line therapy.
